(19) United States
(12) Patent Application Publication
(10) Pub. No.: US 2023/0310320 A1
(43) Pub. Date: Oct. 5, 2023

(54) SOLUBLE MICROCARRIER, METHOD FOR MANUFACTURING AND METHOD OF USE THEREOF

(71) Applicant: National Taiwan University of Science and Technology, TAIPEI CITY (TW)

(72) Inventors: Hsieh-Chih TSAI, New Taipei City (TW); Shuian-Yin LIN, Hsinchu County (TW); Ming-Chien YANG, Taipei City (TW); Chun-Chiang HUANG, New Taipei City (TW); Yu-Hsuan LIN, Taipei City (TW)

(21) Appl. No.: 17/662,847

(22) Filed: May 11, 2022

(30) Foreign Application Priority Data
Feb. 11, 2022 (TW) 111105056

(51) Int. Cl. A61K 9/16

(52) U.S. Cl. CPC A61K 9/1658; A61K 9/1617; A61K 9/1635

(57) ABSTRACT

The present disclosure provides a soluble microcarrier, including soluble polymer including a plurality of soluble monomers binding to each other with a reducing crosslinking agent. The soluble microcarrier of present disclosure facilitates the attachment of cells, and reducing agents can facilitate the detachment of cells. When the soluble microcarrier is in contact with a reducing agent, the soluble microcarrier degrades.

### CROSS - REFERENCE TO RELATED APPLICATION

This application claims priority to Taiwan Application Serial Number 111105056, filed on Feb. 11, 2022, which is herein incorporated by reference in its entirety.

### BACKGROUND

### Field of Invention

The present invention relates to a microcarrier, method for manufacturing and method of use thereof. More particularly, the present invention relates to a soluble microcarrier, method for manufacturing and method of use thereof.

### Description of Related Art

Microcarrier has been thought to be the best of technology to achieve the high density stem cells expansion and used for regenerative medicine. The size of microcarrier is typically a diameter 100-400 µm that offer the high surface area for cell expansion. Traditional microcarrier has been design with high cell attachment and proliferation rate as the targeting. However, the yield of cells recovery was low and makes the harsh cell manufacturing process. The cell harvesting step is technically challenging for cell culture from microcarriers.

Recently, Corning® has developed a dissolvable microcarrier which cross-linked with calcium ion with surface coated with Synthemax® II substrate. The cell harvesting of dissolvable microcarrier can be achieved by adding the EDTA, pectinase and trypsin.

In addition, the use of non-enzymatic detachment methods can avoid cell damage and changing in immune profile. Temperature-induced detachment is a non-invasive behavior, and temperature-sensitive material surface was grafted onto petri dishes or modified on the microcarrier surface. When the temperature is higher than the lower critical solution temperature (LCST), the microcarrier is hydrophobic and can adsorb cells; when the temperature is higher than the LCST, the microcarrier is transformed from hydrophobic to hydrophilic and random coiling, so that the cells detach from the microcarrier. Thermal induction has been used for two-dimensional (2D) cell culture, sensitive materials include pluronic, methylcellulose (MC), and poly(N-isopropylacrylamide) (PNIPAM). However, this detachment method (thermal induction) is time-consuming or less efficient than the enzymatic detachment method.

Therefore, based on the above defect, the related art really needs to be improved.

### SUMMARY

The present disclosure provides a soluble microcarrier, comprising soluble polymer comprising a plurality of soluble monomers binding to each other with a reducing crosslinking agent.

In some embodiments, the reducing crosslinking agent comprises binding to a hydroxyl group, an amine group, a thiol group, or a carboxylic acid group of the soluble polymer.

In some embodiments, the reducing crosslinking agent comprises disulfide bond crosslinking agent, or diselenide bond crosslinking agent.

In some embodiments, the disulfide bond crosslinking agent comprises 3,3′-dithiodipropionic acid di (N-hydroxysuccinimide ester) (DTSP), 3,3′-dithiobis(sulfosuccinimidylpropionate (DTSSP), cysteine, or dithiobis (succinimidyl propionate)(DSP).

In some embodiments, the diselenide bond crosslinking agent comprises 3,3′-Dithiodipropionic acid di(N-hydroxysuccinimide ester), 3,3′-diselanediyldipropionic acid, 2,2′-diselanediylbis(ethan-1 -amine), 2,2′-diselanediylbis(ethan-1-ol), or a combination thereof.

In some embodiments, the soluble polymer comprises cellulose, collagen, gelatin, sodium alginate, chitosan, hyaluronic acid, fruit acid, or a combination thereof.

In some embodiments, a weight ratio of the soluble polymer and the reducing crosslinking agent is from 1:0.08 to 1:0.8.

In some embodiments, soluble microcarrier further comprises a thermosensitive polymer encompassing the soluble polymer.

In some embodiments, the thermosensitive polymer comprising poly(N-isopropylacrylamide)(PNIPAM), poly(N,N diethylacrylamide)(PDEAAM), poly(N-vinylcaprolactam)(PVCL), poly(2-isopropyl-2-oxazoline)(PIOZ), poloxamer, or a combination thereof.

In some embodiments, the thermosensitive polymer further comprises acrylic acid (AAC), allylamine (ALA), acrylamide (AAm), [2-(Methacryloyloxy)ethyl]dimethyl-(3-sulfopropyl)ammonium hydroxide (DMAPS), 2-(Diethylamino)ethyl methacrylate (DEAEMA), 2-Hydroxyethyl methacrylate (HEMA), or a combination thereof.

In some embodiments, the thermosensitive polymer is poly(N-isopropylacrylamide)-co allylamine (P(NIPAM-co-ALA)).

In some embodiments, a weight percentage of the ALA to P(NIPAM-co-ALA) is from 1% to 15%.

In some embodiments, the thermosensitive polymer is bound to an outer surface of the soluble polymer by the reducing crosslinking agent.

In some embodiments, the thermosensitive polymer is physically bound to an outer surface of the soluble polymer.

The present disclosure also provides a method for manufacturing a soluble microcarrier, comprising steps of: providing a soluble polymer; and processing the soluble polymer and a reducing crosslinking agent with a mixing process, wherein when the soluble polymer is in contact with the reducing crosslinking agent, crosslinking occurs to obtain the soluble microcarrier.

In some embodiments, the step of providing the soluble polymer comprises: heating a plurality of soluble monomers to a liquid state; mixing an oil and a surfactant to obtain a mixed solution; mixing the mixed solution and the plurality of soluble monomers to obtain a water-in-oil emulsion; and cooling the water-in-oil emulsion to shape to obtain the soluble polymer.

In some embodiments, the method further comprises providing a thermosensitive polymer; and mixing the soluble microcarrier and the thermosensitive polymer to obtain a soluble-thermosensitive microcarrier.

In some embodiments, the mixing process comprises microfluidics, titration, electrospinning, emulsion polymerization, film emulsification, or a combination thereof.

The present disclosure also provides a method of using a soluble microcarrier as above mentioned, wherein when the soluble microcarrier is in contact with a reducing agent, the soluble microcarrier degrades.

The present disclosure also provides a method of using a soluble microcarrier as above mentioned, wherein when the soluble microcarrier is in contact with a reducing agent, contacts a lower critical solution temperature, contacts the reducing agent and then contacts the lower critical solution temperature, or contacts the lower critical solution temperature and then contacts the reducing agent, the soluble microcarrier degrades.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a schematic view of manufacturing microcarrier according to one embodiment of the present disclosure.

FIG. 2 is a ¹H-NMR spectrum of thermosensitive polymer according to one embodiment of the present disclosure.

FIG. 3 is a line chart of the lowest criticality of thermosensitive polymers with different ALA ratios at different temperatures according to one embodiment of the present disclosure.

FIG. 4 is a bar chart of the water contact angle of thermosensitive polymers with different ALA ratios at different temperatures according to one embodiment of the present disclosure.

FIG. 5 is a ¹H-NMR spectrum of DSeDPA diselenide bond crosslinking agent according to one embodiment of the present disclosure.

FIG. 6 is a ¹H-NMR spectrum of DSeDPA-NHS diselenide bond crosslinking agent according to one embodiment of the present disclosure.

FIG. 7 is a Raman spectrum of diselenide bond crosslinking agent according to one embodiment of the present disclosure.

FIG. 8 is a Fourier-transform infrared spectroscopy (FT-IR) spectrum of diselenide bond crosslinking agent according to one embodiment of the present disclosure.

FIG. 9 is a Raman spectrum of a plurality of soluble microspheres according to one embodiment of the present disclosure.

FIG. 10 is a Raman spectrum of a plurality of soluble-reduceable microspheres according to one embodiment of the present disclosure.

FIG. 11 is a FT-IR spectrum of soluble microsphere (Gms) before and after crosslinking according to one embodiment of the present disclosure.

FIG. 12 is a FT-IR spectrum of a plurality of soluble microspheres according to one embodiment of the present disclosure.

FIG. 13 is a FT-IR spectrum of different types of soluble-reduceable microspheres according to one embodiment of the present disclosure.

FIGS. 14A to 14I are scanning electron microscope (SEM) images of a plurality of microspheres according to one embodiment of the present disclosure; scale bars are all 100 micrometers (µm), FIGS. 14A, B, C, D, H, I are SEI 15.0 kV, 150x (folds), WD 11.4-12.2, and FIGS. 14D, E, F are SEI 5.0 kV 50 folds, WD 10.6-11.7.

FIG. 15 is a line chart of particle size of soluble-reduceable microsphere (Gms-DTSP) with different crosslinking agents’ concentrations after swelling according to one embodiment of the present disclosure.

FIG. 16 is a line chart of swelling ratio of different microspheres after swelling according to one embodiment of the present disclosure.

FIG. 17 is a bar chart of cell viability of madin-darby canine kidney cell (MDCK cell) cultured with 1% ALA thermosensitive polymer according to one embodiment of the present disclosure; n=8.

FIG. 18 is a bar chart of cell viability of MDCK cell cultured with 3% ALA thermosensitive polymer according to one embodiment of the present disclosure; n=8.

FIG. 19 is a bar chart of cell viability of MDCK cell cultured with 5% ALA thermosensitive polymer according to one embodiment of the present disclosure; n=8.

FIG. 20 is a bar chart of cell viability of the soluble microsphere (Gms) and the soluble-thermosensitive microsphere (Gms-pnipam) according to one embodiment of the present disclosure.

FIG. 21 is a bar chart of cell viability of the soluble-reduceable microsphere (Gms-DTSP) and the soluble-reduceable-thermosensitive microsphere (Gms- DTSP-Se-pnipam and Gms-DTPS-pnipam) according to one embodiment of the present disclosure.

FIG. 22 is a fluorescence staining images of different ratios of ALA thermosensitive polymer and MDCK cells according to one embodiment of the present disclosure.

FIG. 23 is a line chart of attachment rate of MDCK cells to the soluble microsphere (Gms) and the soluble-thermosensitive microsphere (Gms-pnipam) according to one embodiment of the present disclosure.

FIG. 24 is an image of the soluble-reduceable-thermosensitive microspheres degrading with GSH according to one embodiment of the present disclosure; scale bar is 100 µm.

FIG. 25 is an image of the soluble-reduceable-thermosensitive microspheres degrading with L-cysteine according to one embodiment of the present disclosure; scale bar is 100 µm.

FIG. 26 is an image of the soluble-reduceable-thermosensitive microspheres degrading with DTT according to one embodiment of the present disclosure; scale bar is 100 µm.

FIG. 27 is a line chart of attachment rate of MDCK cells on the soluble-reduceable-thermosensitive microspheres according to one embodiment of the present disclosure; scale bar is 100 µm.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

Further, spatially relative terms, such as “beneath,” “over” and the like, may be used herein for ease of description to describe one element or feature’s relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

In some embodiments, preparation of a soluble-reduceable microcarrier or a soluble microcarrier includes mixing a soluble polymer and a crosslinking agent. When the soluble polymer is in contact with the crosslinking agent, crosslinking occurs to obtain the soluble-reduceable microcarrier or the soluble microcarrier. In one embodiment, the microcarrier can be form including, but not limited to in approximately spherical microsphere. In some examples, a particle size of the microspheres in the dry state is between 100 to 300 micrometers (µm), and the spheres are suitable for the growth of adherent cells, for example, 110 µm, 120 µm, 140 µm, 200 µm, 220 µm, 250 µm, 280 µm, or any value between any two of these values. In some examples, after the microcarrier is immersed in the medium, the spherical shape becomes complete, and the particle size is between 150 to 400 µm, such as 160 µm, 170 µm, 180 µm, 190 µm, 200 µm, 250 µm, 300 µm, 310 µm, 320 µm, 330 µm, 340 µm, 350 µm, 370 µm, or any value between any two of these values.

The charge and hydrophilic properties of the surface of the microcarrier affect the cell attachment behavior. Positive electrochemical groups, such as amine groups (—NH₂), have better cell adhesion than that of negative electrochemical groups of carboxylic acid groups (—COOH). In addition, the surface is slightly hydrophilic and has better protein adsorption properties than that of hydrophobic (water contact angle>90°) and superhydrophobic (water contact angle>150°).

In one embodiment, the soluble polymer includes cellulose, collagen, gelatin, sodium alginate, chitosan, hyaluronic acid, fruit acid, or a combination thereof. In one embodiment, gelatin consists of 85% to 92% protein, mineral salts and water. A water-soluble mixture was extracted from extracellular matrix collagen in animal skin, bone and connective tissue, and highly biocompatible, biodegradable and non-toxic macromolecules are composed of 300 to 4000 amino acid groups of heterogeneous single chains and multi-chain polypeptide composition. Type A gelatin was obtained by acid hydrolysis of pork rind (pH 3.8 to pH 6.0; isoelectric point 6 to 8); type B gelatin was obtained by alkaline hydrolysis of animal bone and skin (pH 5.0 to pH 7.4; isoelectric point 4.7 to 5.3). Gelatin has a unique amino acid sequence consisting of three parallel left-handed α chains. Each chain is composed of a repeating amino acid sequence Gly-Xaa-Yaa (Gly: glycine, Xaa: proline, Yaa: hydroxyproline), which is easily soluble in high temperature aqueous solvents and forms a gel after cooling. The sol-gel phase transition occurs when the temperature is at the upper critical solution temperature (UCST) of 30° C. to 35° C., which is a reversible gelation phenomenon. In one embodiment, collagen is the main structural protein in the extracellular matrix of many tissues, and collagen is rich in arginine-glycine-aspartic acid (RGD) sequence, which promotes cell adhesion and proliferation.

In one embodiment, an oil and a surfactant are mixed to obtain a mixed solution. In one embodiment, the oil includes mineral oil, stearic acid, cottonseed oil, oleyl alcohol, white wax oil, or a combination thereof. In one embodiment, surfactant includes sorbitan monooleate (span 80), hydroxylated lanolin, polyoxythylene sorbitol beeswax derivative, porpylene glycol fatty acid ester, propylene glycol monolaurate, di(ethylene glycol) monooleate, sodium lauryl ether sulfate (2EO), polyoxythylene sorbitol beeswax derivative, diethylene glycol distearate), or a combination thereof. In one embodiment, a hydrophilic-lipophilic balance (HLB) value of 0 indicates a completely lipophilic molecule, while the larger the value, the more hydrophilic. In some examples, the appropriate surfactant is chosen for water-in-oil gelatin emulsion systems based on the HLB between 3 to 5, for example, 3.2, 3.4, 3.6, 3.8, 4.0, 4.2, 4.4, 4.6, 4.8, or any value between any of these values; in which the HLB of sorbitan monooleate 80 is 4.3, which is relatively lipophilic, and is suitable for dispersing in the oil phase and avoiding the merging of droplets in the water phase, thereby improving the stability of the emulsion.

In one embodiment, next, the soluble polymers and water are heated to form a soluble polymer aqueous solution, and then the soluble polymer aqueous solution is slowly added into the mixed solution to obtain a water-in-oil (W/O) emulsion (oil:water v/v from 10:1 to 4:1, including, but not limited to 9:1, 8:1, 7:1, 6:1, 5:1, or any value between any of these values. The amount of oil here should not be too low to avoid poor molding. In one embodiment, the soluble polymers and water are heated so that the soluble polymers or monomer thereof are form in a liquid state. Heating temperature includes, but is not limited to 30° C. to 90° C., for example, 40° C., 50° C., 60° C., 70° C., 80° C., or any value between any of these values.

In one embodiment, next, the W/O emulsion is cooled to shape (or cold set) the microspheres, crosslinking agent is added and stirred for crosslinking reaction until the microspheres solidify. The terms “shape” described herein refers to the form of stable water in the oil. The terms “solidify” described herein refers to that the shape of the water droplet or microsphere does not change anymore; the emulsified material is modified with a small amount of surface and characteristics, and the shape of the material will no longer be changed according to the modified characteristics. In one embodiment, the temperature of the cooling the W/O emulsion described herein is less than the aforementioned heating temperature. In one embodiment, regarding preparing the soluble-reduceable microsphere, crosslinking agent is reducing crosslinking agent, the reducing crosslinking agent bonds to the hydroxyl, amine, thiol, or carboxylic acid group of the soluble polymer. In some examples, reducing crosslinking agent includes, but is not limited to disulfide bond crosslinking agent or diselenide bond crosslinking agent. Disulfide bond crosslinking agent includes, but is not limited to DTSP, DTSSP, cysteine, or DSP. Diselenide bond crosslinking agent includes, but is not limited to DSeDPA-NHS, 3,3′-diselanediyldipropionic acid, 2,2′-diselanediylbis(ethan-1 -amine), 2,2′-diselanediylbis(ethan-1-ol), or a combination thereof. In another embodiment, in the preparation of dissolvable microcarriers, the crosslinking agent includes a zero-length crosslinking agent and a non-zero length crosslinking agent. The zero-length crosslinking agent is removed after the crosslinking of the catalytically degradable polymer is completed. The zero-length crosslinking agent includes, but is not limited to carbodiimide (EDC), N,N′-dicyclohexylcarbodiimide (DSC), N,N-dicyclohexylcarbodiimide (DCC), or a combination thereof. The non-zero length crosslinking agent will eventually be incorporated into the polymer network, and the crosslinking agent will react with the degrading polymer to form covalent bonds between the degrading polymers. The non-zero length crosslinking agent includes, but is not limited to formaldehyde, glutaraldehyde, acrylamide, isocyanate, gardenia, DTSP, DSeDPA-NHS, BSSS, DSG, sulfo-EGS, DSS, EGS, BS2G, DTSSP, DST, BSOCOES, DPDPB, sulfo DST, or DSP.

In one embodiment, next, after microsphere solidification and crosslinking reaction, the oil phase is removed and the microspheres are washed, and then the microspheres are dried to obtain soluble-reduceable microspheres (Gms-DTSP) or soluble microspheres (Gms). In some examples, after the crosslinking reaction, the oil phase is removed by a suction filter device, and then the microspheres are washed several times with acetone/water solution (v/v, 5:1~1:1, such as 4:1, 3:1 or 2:1). Finally, the microspheres are freeze-dried. In some examples, the ratio of acetone/water solution herein can be fixed or decreased in sequence. Oil can be quickly removed under high concentration of acetone/water solution, but it cannot be cleaned with acetone, which will damage the surface or shape of the microspheres.

In one embodiment, a weight ratio of the soluble polymer to the reducing crosslinking agent is 1:0.08 to 1:0.8.

In one embodiment, a weight ratio of the soluble polymer to the reducing crosslinking agent is 1:0.32 to 1:0.8 ◦

In some embodiments, the dried soluble-reduceable microspheres (Gms-DTSP) are measured by an elemental analyzer, and a weight ratio of the gelatin to DTSP is about 99:1 to 1.86:1, for example, about 90:1 to 2:1, 80:1 to 2:1, 70:1 to 2:1, 60:1 to 2:1, 50:1 to 2:1, 40:1 to 2:1, 30:1 to 2:1, 20:1 to 2:1, 10:1 to 2:1, 7:1 to 2:1, 5.67:1 to 1.86:1, 5:1 to 1.86:1, 4:1 to 1.86:1, 3:1 to 1.86:1, or any value between any two of these values.

In some embodiments, The thermosensitive polymer (copolymer) is obtain by the preparation method including polymerizing a thermosensitive macromolecular compound and a hydrophilic monomer by a free radical polymerization. In one embodiment, thermosensitive macromolecular compound includes, but is not limited to PNIPAM, PDEAAM, PVCL, PIOZ, poloxamer, or any value between any two of these values. In one embodiment, the hydrophilic monomer includes, but is not limited to AAC, ALA, Aam, DMAPS, DEAEMA, HEMA, or a combination thereof. In one embodiment, the free radical polymerization includes, but is not limited to SFRP, ATRP, or RAFT. In one embodiment, the hydrophilic monomer includes, but is not limited to AAC, ALA, Aam, DMAPS, DEAEMA, HEMA, or a combination thereof. In some examples, thermosensitive polymer includes N-isopropylacrylamide and allylamine, in which a weight percentage of allylamine to the thermosensitive polymer is 1% to 5%, for example, 2%, 3%, 4%, or any value between any two of these values.

In one embodiment, the thermosensitive macromolecular compound, the hydrophilic monomer, an initiator, and a chain transfer agent are dissolved in an organic solvent by reversible addition fragmentation chain transfer polymerization (RAFT), and placed on the ultrasonic shaker to dissolve to obtain a mixture. After the mixture is added to the reaction flask and purged with nitrogen, the polymerization is processed with heating and continuously stirring. The device condenser tube above the reaction flask maintains the system reflux to avoid that the temperature is too high and the reactant loss is due to heat and volatilize. After the viscosity of the mixture heated in the reaction flask is no longer thickened, the reaction flask is put into liquid nitrogen to terminate the reaction to obtain a polymerization solution. In some examples, heating temperature is from 50° C. to 90° C., for example, 60° C., 65° C., 70° C., 75° C., 80° C., 85° C., or any value between any two of these values. In some examples, the reaction time is from 4 hrs. to 48 hrs., for example, 6 hrs., 8 hrs., 10 hrs., 15 hrs., 20 hrs., 25 hrs., 30 hrs., 35 hrs., 40 hrs., 45 hrs., or any value between any two of these values. In some embodiments, common chain transfer agents include mercaptans, dodecyl mercaptan (DDM), or alkyl halides such as carbon tetrachloride. The chain transfer agents are also known as modifiers and control agents.

In one embodiment, the polymerization solution is re-precipitated in cold ether to ensure removal of the unreacted monomers and the chain transfer agent. A solid is obtained after precipitation and dried to remove ether. Next, a dialysis membrane is used for the purification step, the dried solid is dialyzed and purified in water to obtain a semi-finished product, and the water is removed from the semi-finished product to obtain a dry thermosensitive polymer, or called thermosensitive block copolymer.

In some embodiments, preparing soluble-reduceable-thermosensitive microsphere (Gms-DTSP-pnipam) or soluble-thermosensitive microsphere (Gms- pnipam) by physical method includes mixing the soluble-reduceable microcarrier and the thermosensitive polymer to obtain the soluble-reduceable-thermosensitive microcarrier; or mixing the soluble microcarrier and the thermosensitive polymer to obtain the soluble -thermosensitive microcarrier. In one embodiment, after preparing a thermosensitive polymer aqueous solution, the soluble-reduceable microspheres (Gms-DTSP) or the soluble microspheres (Gms) are added and stirred at a low temperature until the soluble microspheres are covered with thermosensitive polymer. The microspheres after modified are washed to remove residual thermosensitive polymer, and then dried to remove water to obtain soluble-reduceable-thermosensitive microspheres (Gms-DTPS-pnipam) or soluble-thermosensitive microspheres (Gms-pnipam). In some examples, the microspheres are stirred at a low temperature of 0° C. to 15° C., for example, 1° C., 2° C., 3° C., 4° C., 5° C., 6° C., 10° C., 12° C., 14° C., or any value between any two of these values. In some examples, the microspheres are dried to remove water. In one embodiment, the physical coating of thermosensitive polymer on the surface of the microcarrier is achieved through intermolecular forces, for example, Van der Waals force, secondary bond includes, but is not limited to hydrogen bond and so forth.

In some embodiments, preparing diselenide bond crosslinking agent includes 10 millimoles (mmol) selenium (Se) powder is prepared in 3 mL water and kept under nitrogen environment. In some examples, the selenium-containing water is injected into a three-necked reaction flask and kept in nitrogen. Subsequently, 20 mmol of sodium borohydride (NaBH₄) is slowly dropped into the selenium-containing water in 8 mL water, and stirred until colorless to completely dissolve the selenium powder to obtain a first mixed solution. Then, an equal amount of 10 mmol selenium powder is added to the first mixed solution and is heated until it turns reddish brown to obtain a second mixed solution. In some examples, the heating temperature ranges from 80° C. to 130° C., for example, 85° C., 90° C., 95° C., 100° C., 105° C., 110° C., 115° C., 120° C., 125° C., or any value between any two of these values. Next, 20 mmol of 3-chloropropionic acid is added to the second mixed solution and stirred under a nitrogen environment at room temperature to obtain a third mixed solution. The third mixed solution is exposed to the atmosphere and stirred, followed by filtration to remove unreacted material, yielding a yellow supernatant. The yellow supernatant is adjusted to pH 3.5 with 1 M hydrochloric acid, and extracted with anhydrous ethyl acetate to obtain an upper organic layer. The upper organic layer is washed and extracted twice with water, and the water and ethyl acetate are removed to obtain DSeDPA.

In one embodiment, 1.2 mM DSPA is dissolved in 5 mL of anhydrous tetrahydrofuran and then dropped into a container under nitrogen environment. Subsequently, 2.88 mmol of N-hydroxysuccinimide (NHS) is added to the container and stirred to obtain an initial solution. Then, 2.88 mmol of carbodiimide (EDC) is dissolved in 5 mL of anhydrous tetrahydrofuran, and dropped into the initial solution at low temperature, and the reaction speed is controlled to avoid too fast. In some examples, the low temperature is ranges from 0° C. to 20° C., for example, 5° C., 10° C., 15° C., or any value between any two of these values. Next, the initial solution containing tetrahydrofuran is stirred at room temperature to complete the reaction, and DSeDPA-NHS is obtained after filtering impurities and removing tetrahydrofuran. DSeDPA-NHS can also be collected and stored after being dried in a vacuum oven for 24 hours.

In some embodiments, preparing soluble-reduceable-thermosensitive microsphere (Gms-DTSP-Se-pnipam) by chemical method includes mixing the soluble-reduceable microcarrier, a reducing crosslinking agent, and the thermosensitive polymer to obtain the soluble-reduceable-thermosensitive microcarrier; or mixing the soluble microcarrier, the reducing crosslinking agent, and the thermosensitive polymer to obtain soluble-thermosensitive microcarrier. In one embodiment, after preparing the thermosensitive polymer aqueous solution, 0.18 mM DSeDPA-NHS and soluble-reduceable microspheres (Gms-DTSP) or soluble microspheres (Gms) are added and stirred at a low temperature until the microspheres are covered and chemically bonded with thermosensitive polymer. In some examples, the microspheres are stirred under 0° C. to 15° C., for example, 1° C., 2° C., 3° C., 4° C., 5° C., 6° C., 10° C., 12° C., 14° C., or any value between any two of these values. The microspheres after bonding are washed to remove the residual thermosensitive polymer, and then dried to remove water to obtain the soluble-reduceable-thermosensitive microspheres (Gms-DTSP-Se-pnipam) or the soluble-thermosensitive microspheres (Gms-Se-pnipam).

In some examples, in the preparation of soluble-reduceable-thermosensitive microsphere (Gms-DTSP-Se-pnipam) by chemical method, a weight concentration percentage (wt%) of pnipam, Se, Gms corresponds to: pnipam 3% to 10%, Se 1% to 3%, Gms 87% to 96%. A weight by part of pnipam, Se, Gms are 3~10:1~3:87~96, for example, a weight by part of pnipam is 3, 4, 5, 6, 7, 8, 9, 10, or any value between any two of these values; a weight by part of Se is 1, 2, 3, or any value between any two of these values; a weight by part of Gms is 87, 88, 89, 90, 91, 92, 93, 94, 95, 96, or any value between any two of these values.

Although a series of operations or steps are used below to describe the method disclosed herein, an order of these operations or steps should not be construed as a limitation to the present invention. For example, some operations or steps may be performed in a different order and/or other steps may be performed at the same time. In addition, all shown operations, steps and/or features are not required to be executed to implement an embodiment of the present invention. In addition, each operation or step described herein may include a plurality of sub-steps or actions.

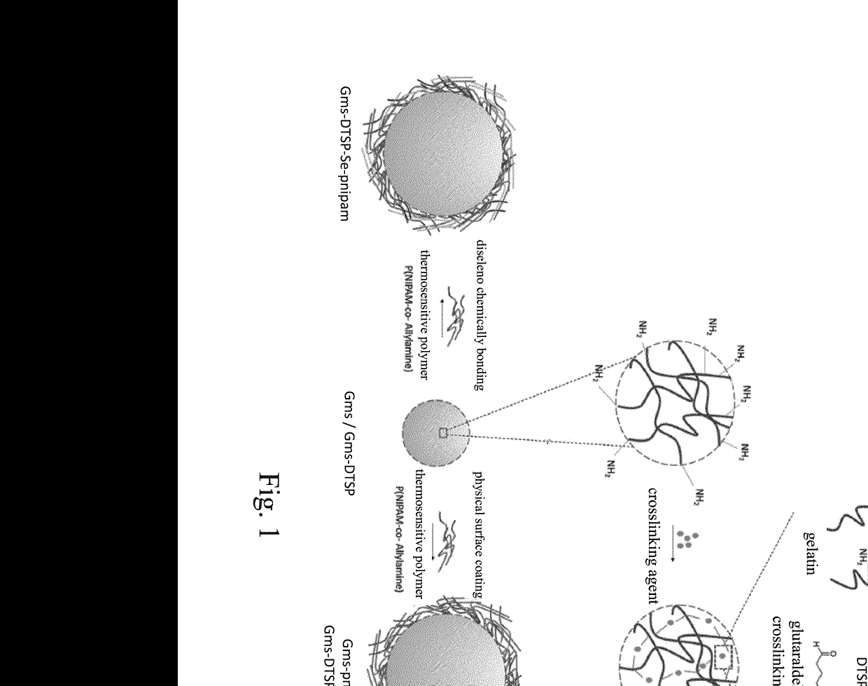

FIG. 1 is a schematic view of manufacturing microcarrier according to one embodiment of the present disclosure.

### Preparation Example 1 Preparing Soluble-reduceable Microspheres

Mineral oil was mixed with surfactant (sorbitan monooleate 80) to form a mixed solution. Gelatin and water were heated to prepare 5 mL of 0.25 g/mL aqueous gelatin solution, and then the aqueous gelatin solution was slowly dropped into the mixed solution to obtain a water-in-oil (W/O) emulsion (oil:water v/v, 7:1). The W/O emulsion was rapidly cooled to shape the microsphere, 0.1 g (gram) to 1 g of the disulfide bond crosslinking agent (DTSP) was added and stirred for crosslinking reaction until the microspheres were solidified (DTSP herein is about 0.25 mM to 1.2 mM). After the crosslinking reaction, the oil phase was removed and washed, and then the microspheres were dried to obtain soluble-reduceable microspheres (Gms-DTSP).

Measured by an elemental analyzer, the weight ratio of gelatin to DTSP is about 99:1~65:35 in the dried soluble-reduceable microspheres (Gms-DTSP).

### Preparation Example 2 Preparing Soluble Microsphere

Mineral oil was mixed with surfactant (sorbitan monooleate 80) to form a mixed solution. Gelatin and water were heated to prepare 0.25 g/mL aqueous gelatin solution, and then the aqueous gelatin solution was slowly dropped into the mixed solution to obtain a water-in-oil (W/O) emulsion (oil:water v/v, 7:1). The W/O emulsion was rapidly cooled to shape the microspheres for a period of time, glutaraldehyde was added and stirred in a period of time for crosslinking reaction until the microspheres were solidified. After the crosslinking reaction, the oil phase was removed and the microspheres were washed, and then the microspheres were dried to obtain soluble microspheres (Gms).

### Preparation Example 3 Preparing Thermosensitive Polymer

NIPAM, ALA, initiator (2,2′-azobis(2-methyl-propionitrile), AIBN), chain transfer agent (4-cyano-4-(phenylcarbonothioylthio) acid, CTA) were dissolved in 1,4-dioxane (1,4-Dioxane) by reversible addition fragmentation chain transfer polymerization (RAFT), and placed on the ultrasonic shaker to dissolve to obtain a mixture. After the mixture was slowly added to the reaction flask and purged with nitrogen, the polymerization was processed with heating and continuously stirring. The device condenser tube above the reaction flask maintained the system reflux to avoid that the temperature is too high and the reactant loss is due to heat and volatilize. After the viscosity of the mixture heated in the reaction flask was no longer thickened, the reaction flask was put into liquid nitrogen to terminate the reaction to obtain a polymerization solution, and the polymerization solution was re-precipitated in cold ether to ensure removal of unreacted monomers (for example, NIPAM, ALA) and chain transfer agent. A light-yellow solid was obtained after precipitation and dried to remove ether. Next, a dialysis membrane (molecular weight cut off (MWCO)=1000) was used for the purification step, the dried light-yellow solid was dialyzed and purified in water to obtain a semi-finished product, and the water was removed from the semi-finished product to obtain a dry thermosensitive polymer P(NIPAM-co -Allylamine), or called thermosensitive block copolymer (yield: about 90%).

### Preparation Example 4 Preparing Soluble-reduceable-thermoSensitive Microsphere (Gms-DTSP-pnipam) - by Physical Method

After preparing 3 wt% P(NIPAM-co-Allylamine) ethanol aqueous solution, the soluble-reduceable microspheres (Gms-DTSP) prepared in Preparation Example 1 was added and stirred at a low temperature of 0~5° C. until the soluble microspheres were covered with P(NIPAM-co-Allylamine). The microspheres after modified were washed to remove residual P(NIPAM-co-Allylamine), and then dried to remove water to obtain soluble-reduceable-thermosensitive microspheres (Gms-DTPS-pnipam).

### Preparation Example 5 Preparing Soluble-thermosensitive Microsphere (Gms-pnipam) - by Physical Method

After preparing 3 wt% P(NIPAM-co-Allylamine) ethanol aqueous solution, the soluble microspheres (Gms) prepared in Preparation Example 2 was added and stirred at a low temperature of 0~5° C. until the soluble microspheres were covered and physically bonded with P(NIPAM-co-Allylamine). The microspheres after modified were washed to remove residual P(NIPAM-co-Allylamine), and then dried to remove water to obtain soluble-thermosensitive microspheres (Gms-pnipam).

### Preparation Example 6 Preparing Diselenide Bond Crosslinking Agent

10 mmol Se powder was prepared in 3 mL water and kept under nitrogen environment. Subsequently, 20 mmol of sodium borohydride (NaBH₄) was slowly dropped into the selenium-containing water in 8 mL water, and stirred until colorless to completely dissolve the selenium powder to obtain a first mixed solution. Then, an equal amount of 10 mmol selenium powder was added to the first mixed solution and was heated until it turns reddish brown to obtain a second mixed solution. 20 mmol of 3-chloropropionic acid was added to the second mixed solution and stirred under a nitrogen environment at room temperature to obtain a third mixed solution. The third mixed solution was exposed to the atmosphere and stirred, followed by filtration to remove unreacted material, yielding a yellow supernatant. The yellow supernatant was adjusted to pH 3.5 with 1 M hydrochloric acid, and extracted with anhydrous ethyl acetate to obtain an upper organic layer. The upper organic layer was washed and extracted twice with water, and the water and ethyl acetate were removed to obtain DSeDPA (yield: 85%).

1.2 mM DSPA was dissolved in 5 mL of anhydrous tetrahydrofuran and then dropped into a container under nitrogen environment. Subsequently, 2.88 mmol of N-hydroxysuccinimide (NHS) was added to the container and stirred to obtain an initial solution. Then, 2.88 mmol of carbodiimide (EDC) was dissolved in 5 mL of anhydrous tetrahydrofuran, and dropped into the initial solution at low temperature, and the reaction speed was controlled to avoid too fast. The initial solution containing tetrahydrofuran was stirred at room temperature to complete the reaction, and DSeDPA-NHS was obtained after filtering impurities and removing tetrahydrofuran. DSeDPA-NHS can also be collected and stored after being dried in a vacuum oven for 24 hours.

### Preparation Example 7 Preparing Soluble-reduceable-thermosensitive Microsphere (Gms-DTSP-Se-pnipam) - by Chemical Method

After preparing 3 wt% P(NIPAM-co-Allylamine) ethanol aqueous solution, 1 wt% DSeDPA-NHS and 96 wt% soluble-reduceable microspheres (Gms-DTSP) prepared in Preparation Example 1 were added and stirred at a low temperature of 0~5° C. until the soluble microspheres were covered and chemically bonded with P(NIPAM-co-Allylamine). The microspheres after bonding were washed to remove residual P(NIPAM-co-Allylamine), and then dried to remove water to obtain soluble-reduceable-thermosensitive microspheres (Gms-DTSP-Se-pnipam).

### Example 1 Synthesis of Thermosensitive Polymer

Thermosensitive polymer was obtained from Preparation Example 3, ALA monomer was polymerized with NIPAM, so that the amine group in the structure had positive electric properties, which is beneficial to the application of cell attachment. The following was divided into three groups of ratios 1% ALA, 3% ALA, 5% ALA. The experimental synthesis ratio formula is shown in Table 1. The synthesized polymer was confirmed by FT-IR and ¹H-NMR to successfully graft and through gel permeation chromatography. Molecular weight was measured by gel permeation chromatography (GPC).

TABLE 1

the synthesis formula of P(NIPAM-co-Allylamine)

| Polymer | Code | NIPAM (mmol) | ALA (mmol) | CTA (mmol) | AIBN (mmol) |
|---|---|---|---|---|---|
| P(NIPAM -co-Ala) | 1% ALA | 29.69 | 0.3 | 0.02 | 0.01 |
| | 3% ALA | 29.07 | 0.9 | | |
| | 5% ALA | 28.49 | 1.5 | | |

### 1.1 ¹H-NMR Identification and Composition Elements

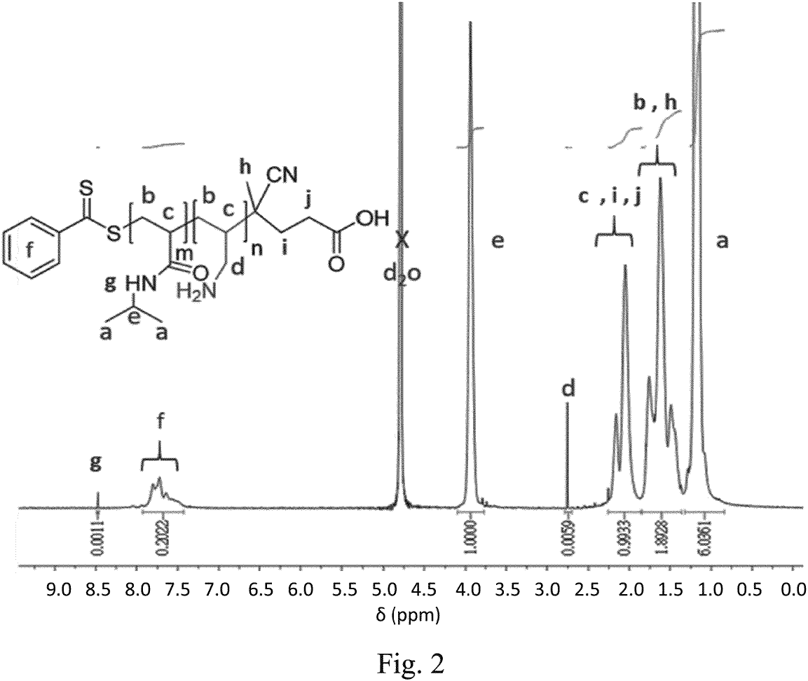

First, the chemical structure of the P(NIPAM-co-Allylamine) polymer was confirmed. The initiator AIBN was added during the reaction, and when the temperature reached 70° C., free radicals began to be generated, which promotes the polymerization of the monomer double bond (C═C). Hydrogen spectrum (¹H-NMR) analysis and identification of molecular structure were processed by nuclear magnetic resonance spectrometer (NMR)(as shown in FIG. 2). The structure corresponds to δ= 0.95 - 1.24 ppm (a, CH₃ from NIPAM), δ= 1.34 -1.75 ppm (b, CH₂ from NIPAM and Ala) (h, CH₂ from CTA), δ= 1.82 - 2.13 ppm (c, CH from NIPAM and Ala) (i, j, CH from CTA), δ= 2.66 ppm (d, CH₂ from Ala), δ= 3.82 ppm (e, CH from NIPAM), δ= 7.32 - 7.79 ppm (f, CH from CTA), δ= 8.38 ppm (g, NH from NIPAM). The graft ratio and composition were calculated by integrating the ¹H-NMR signal (Table 2 below).

TABLE 2

composition ratio of P(NIPAM-co-Allylamine)

| Polymer | Code^{a} | In feed (mol%) | | In copolymer (mol%) | |
|---|---|---|---|---|---|
| | | NIPAM | ALA | NIPAM | ALA |
| P(NIPAM -co-Ala) | 1% ALA | 99 | 1 | 99.82 | 0.14 |
| | 3% ALA | 97 | 3 | 99.75 | 0.24 |
| | 5% ALA | 95 | 5 | 99.68 | 0.31 |

^{a}X% ALA = (mole_{ALA}/mole_{NlPAM})×100%

### 1-2 Molecular Weight Measurement and Identification

The molecular weight and molecular weight distribution of synthesized macromolecules were identified by gel permeation chromatography. Table 3 shows the molecular weight and molecular weight distribution of P(NIPAM-co-Allylamine) thermosensitive polymer measured by gel permeation chromatography. Table 3 shows that the molecular weight dispersity index (PDI) of the polymers have a narrow molecular weight distribution, and the value is mostly from 1.2 to 1.3. The weight-average molecular weights (Mw) of P(NIPAM-co-Allylamine) are 37666, 42803, and 43056, respectively, which proves that the monomers were successfully polymerized into copolymers via RAFT.

TABLE 3

Properties analysis of P(NIPAM-co-Allylamine)

| Code | Mn* (g/mol) | Mw (g/mol) | PDI |
|---|---|---|---|
| 1% ALA | 29739 | 37666 | 1.26 |
| 3% ALA | 32341 | 42803 | 1.32 |
| 5% ALA | 33959 | 43056 | 1.26 |

*Mn : Number-average molecular weight

### 1-3 LCST Measurement

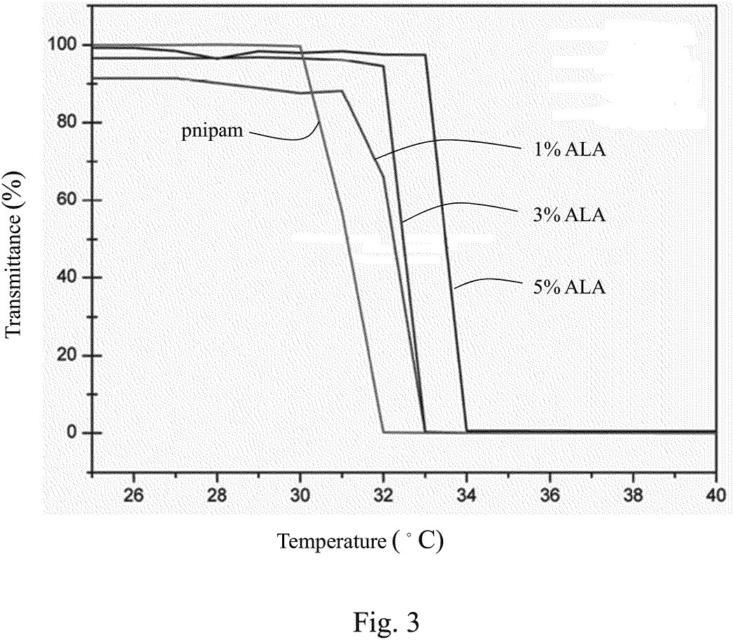

After the monomer ALA was embedded in the NIPAM copolymer, the PNIPAM hydrophobic force affected the LCST because of the increase of the electrostatic repulsion force. The LCST of the copolymer can be measured by ultraviolet-visible (UV/Vis) spectrometer, which is the corresponding temperature under 50% transmittance: when the temperature rises, the copolymer aggregates in large quantities, forming a non-flowing gel state from the solution state, and the light penetration value is relatively low. FIG. 3 is the transmittance rate of PNIPAM and 1%ALA, 3%ALA, 5%ALA at different temperatures, it is known that the LCST of PNIPAM is 31.4° C.

FIG. 3 is the transmittance rate of PNIPAM and 1 %ALA, 3%ALA, 5%ALA at different temperatures, and the LCST of PNIPAM is 31.4° C.; the LCST of 1 %ALA, 3%ALA, 5%ALA are 32.3° C., 32.5° C., 33.4° C., respectively. Thus, LCST increases with ALA ratio. This confirms that the adding of hydrophobic monomers to the copolymer reduces LCST, whereas hydrophilic monomers increase LCST, mainly depending on the hydrophilic and hydrophobic properties of the polymer tails.

### 1-4 Water Contact Angle Test

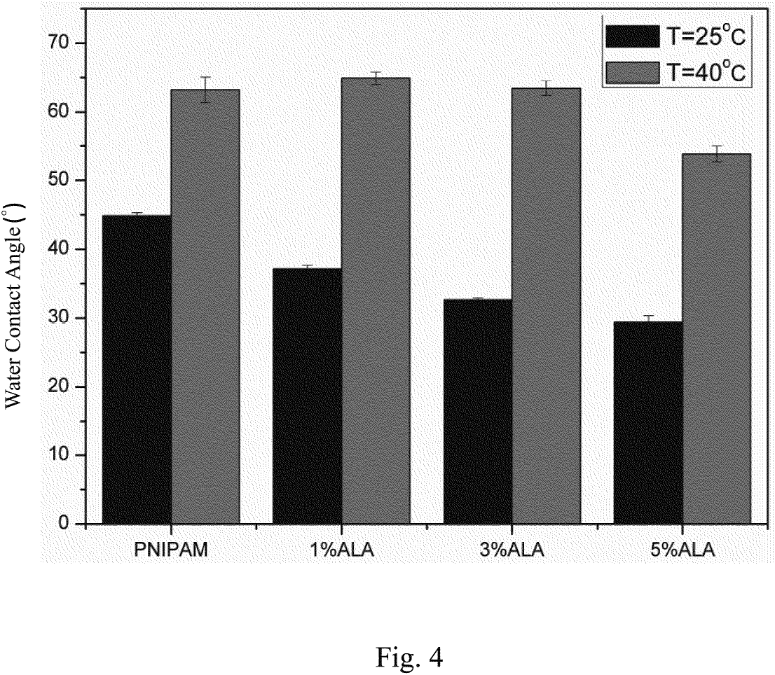

Water contact angle of thermosensitive polymer varies with temperature. Water produces a circular water droplet shape at the gas-liquid interface by its own surface tension. The water contact angle is calculated according to the Young formula: _{Y}sv=_{Y}sl+_{Y}lvcosθ through the image. Please refer to FIG. 4, commercially available PNIPAM is as a control. The water contact angle of PNIPAM at room temperature is 44.83 ± 0.47°, and the water contact angles of 1% ALA, 3% ALA, 5% ALA after embedding ALA are 37.13 ± 0.55°, 32.65 ± 0.28°, 29.4 ± 0.95°, respectively. When the temperature increased from room temperature to 40° C., the water contact angles of PNIPAM, 1 %ALA, 3%ALA, and 5%ALA were 63.23 ± 1.85°, 64.91 ± 0.89°, 63.45 ± 1.04°, 53.83 ± 1.17°, respectively. In the control group, the angle difference of PNIPAM from low temperature to high temperature is 18.4°, and the difference of P(NIPAM-co-Allylamine) after ALA embedded is more than 25°, which confirms that the copolymer has temperature response characteristics. In addition, ALA is a hydrophilic monomer, and 5% ALA at low temperature has a smaller water contact angle and has the most hydrophilic property. With the increase of ALA content in the copolymer, the contact angle is gradually decrease. 1 %ALA and 3%ALA are closer to the hydrophobic contact angle of PNIPAM in the control group at high temperature, which is consistent with slightly hydrophilic (water contact angle < 90°) and has better protein adsorption characteristics, which proves that it is beneficial to cell adsorption and proliferation.

### Example 2 Synthesis of Diselenide Bond Crosslinking Agent

One of the purposes of synthesizing diselenide bonds is to combine P(NIPAM-co-Allylamine) polymer with microspheres by chemical bonding to form thermosensitive microspheres, and compared with the thermosensitive polymer physically coating on the surface of microspheres. The diselenide bond is a redox-sensitive material and is easily broken by environmental changes. The oxygen in the carboxylic acid group (—COOH) at both ends of DSeDPA is nucleophilically attacked by EDC to form a highly reactive intermediate (O-acylisourea), the second intermediate (O-acylisourea) formed by subsequent reaction with NHS is hydrolyzed to DSeDPA-NHS in water, and the DSeDPA-NHS rapidly reacts with the amine group (—NH₂) to generate a stable amide bond. At this time, DSeDPA-NHS reacts with the amine group at the end of P(NIPAM-co-Allylamine) to form a polymer.

### 2.1 ¹H-NMR Identification and Composition Elements

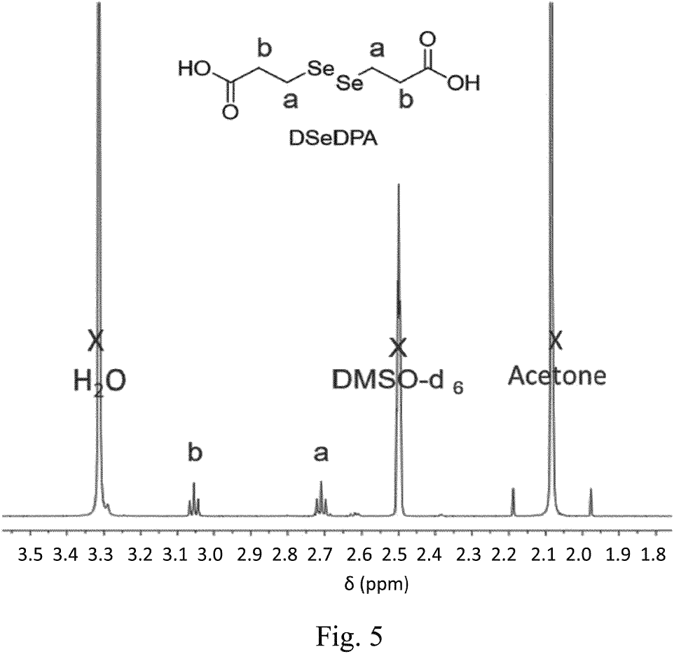

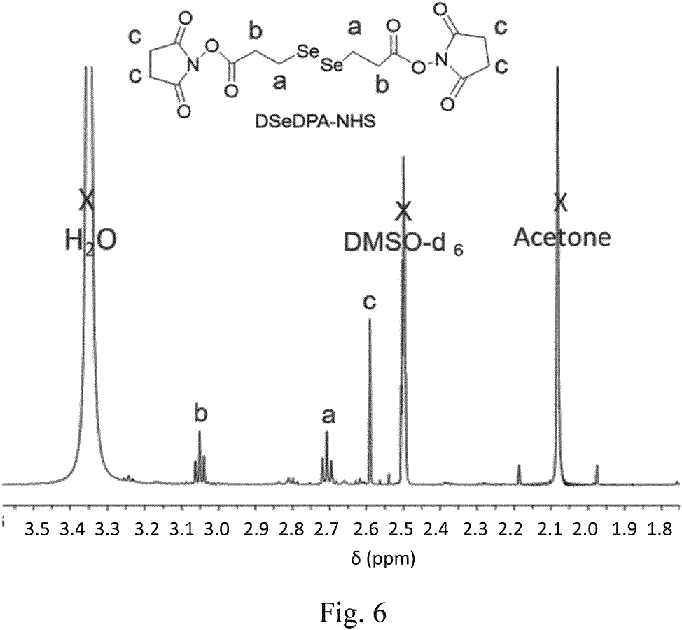

The correctness of the structure was identified by ¹H-NMR. Before DSeDPA activation (as shown in FIG. 5), it was found that corresponds to the structural formula δ= 2.69~2.72 ppm (a); δ= 3.04~3.06 ppm (b). The structural formula of the two points is CH₂, the carboxylic acid group (-COOH) next to point b is pulled and shifted to the left by a large electronegativity, which is consistent with the increase in resonance frequency in NMR and the signal is located in the low-field region. After activation (as shown in FIG. 6), DSeDPA-NHS structural formula δ= 2.69 to 2.71 ppm (a, CH₂ from DSeDPA ); δ= 3.03 to 3.06 ppm (b, CH₂ from DSeDPA ); δ=2.59 ppm (c, CH from NHS). From point c, it can be proved that there are four CH signal intensities on the NHS active group higher than that of points a and b. The above NMR analysis proves that the diselenide bond crosslinking agent DSeDPA-NHS functional group was successfully synthesized.

### 2-2 Raman Spectrum Analysis

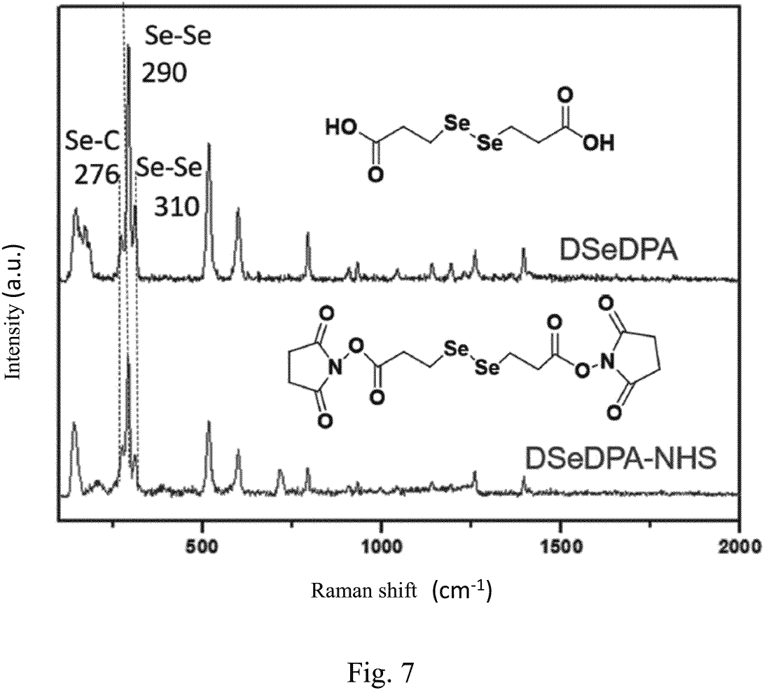

Selenium is an air-sensitive element. FIG. 7 shows the Raman spectrum of the symmetrical structure of selenium. After analysis, Se-Se double-selenide bond signals appear at 290 cm⁻¹ and 310 cm⁻¹, and there are obvious Se-C functional groups around 276 cm⁻¹. Therefore, it is proved that the activation of EDC can improve the activity and reaction efficiency of carboxylic acid, and connect NHS to carboxylic acid group (—COOH). The DSeDPA-NHS was successfully synthesized, and the presence of selenium element in the structure was confirmed by Raman.

### 2-3 FT-IR Analysis

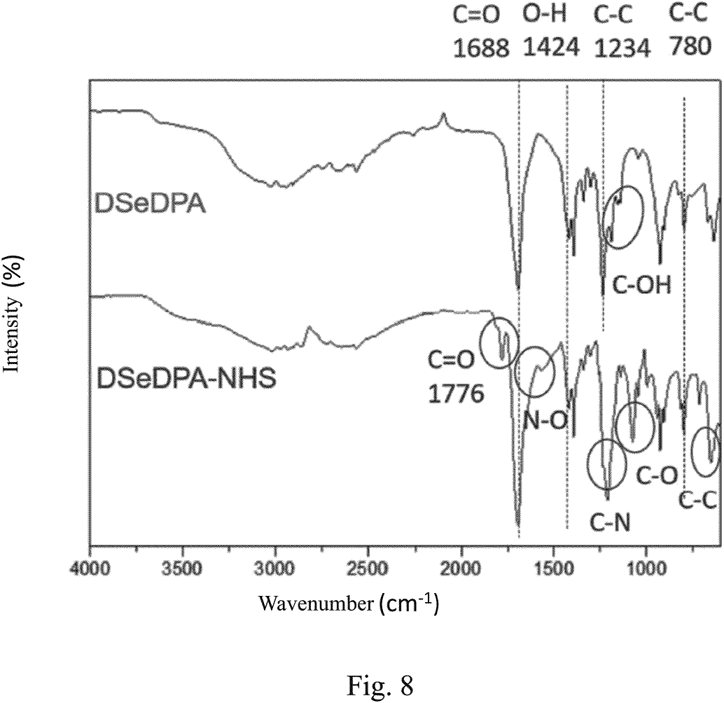

The structure of inorganic functional groups can be more accurately observed by using FT-IR. From the spectrum analysis in FIG. 8, it can be seen that after the DSeDPA carboxylic acid group was combined with NHS before activation, it belongs to the C═O functional group at 1688 cm⁻¹ . After activation, it was pulled because of NHS binding and changing to a peak at 1776 cm⁻¹. In addition, the spectra of other functional groups signal N-O, C-N, C-O molecular vibrations are obviously different, which means the reaction was successful.

### Example 3 Identification of Gelatin Microsphere

Gelatin is soluble in hot water but not in cold water, but it needs to be at 37° C. for human cell culture. Therefore, it is necessary to stabilize the network crosslinking and add crosslinking agent to obtain microspheres through emulsification reaction. The microspheres prepared with reducing crosslinking agent are soluble-reduceable microspheres (Gms-DTSP, as in Preparation Example 1), and the microspheres prepared with non-reducing crosslinking agent are soluble microspheres (Gms, as in Preparation Example 2). Then, the thermosensitive polymer is bonded to the surface of the microspheres by physical coating or chemical bonding to obtain the physically coating soluble-reduceable-thermosensitive microsphere (Gms-DTPS-pnipam, as in Preparation Example 4) and the soluble-thermosensitive microspheres (Gms-pnipam, as in Preparation Example 5), and chemically bonded soluble-reduceable-thermosensitive microspheres (Gms-DTSP-Se-pnipam, as in Preparation Example 7), respectively.

### 3.1 Raman Spectrum Analysis

Each group of microspheres was analyzed by Raman instrument and divided into two groups according to the crosslinking agent:
First group: soluble microsphere (Gms, as in Preparation Example 2), physically coating soluble-thermosensitive microsphere (Gms-pnipam, as in Preparation Example 5);
Second group: soluble-reduceable microspheres (Gms-DTSP, as in Preparation Example 1), chemically bonded soluble-reduceable-thermosensitive microsphere (Gms-DTSP-Se-pnipam, as in Preparation Example 7), physically coating soluble-reduceable-thermosensitive microsphere (Gms-DTPS-pnipam, as in Preparation Example 4)

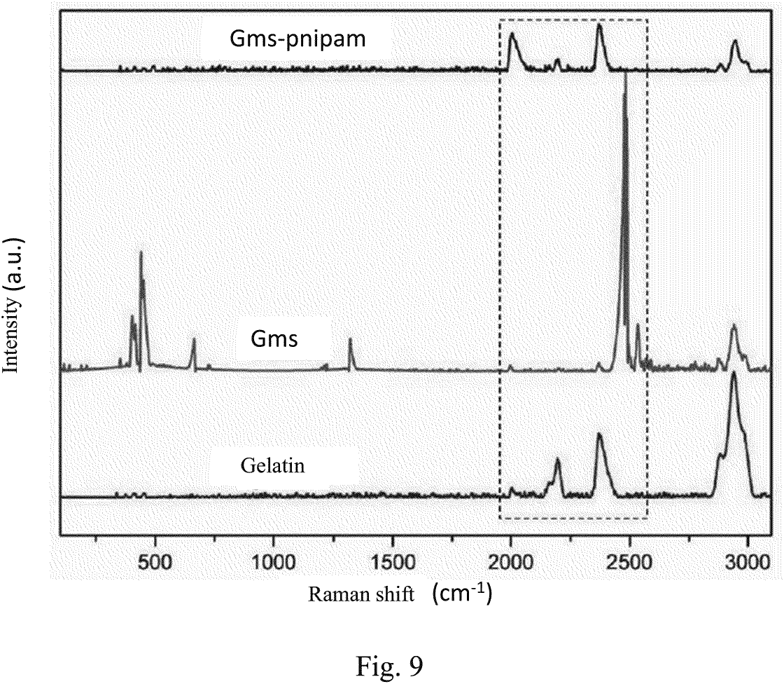

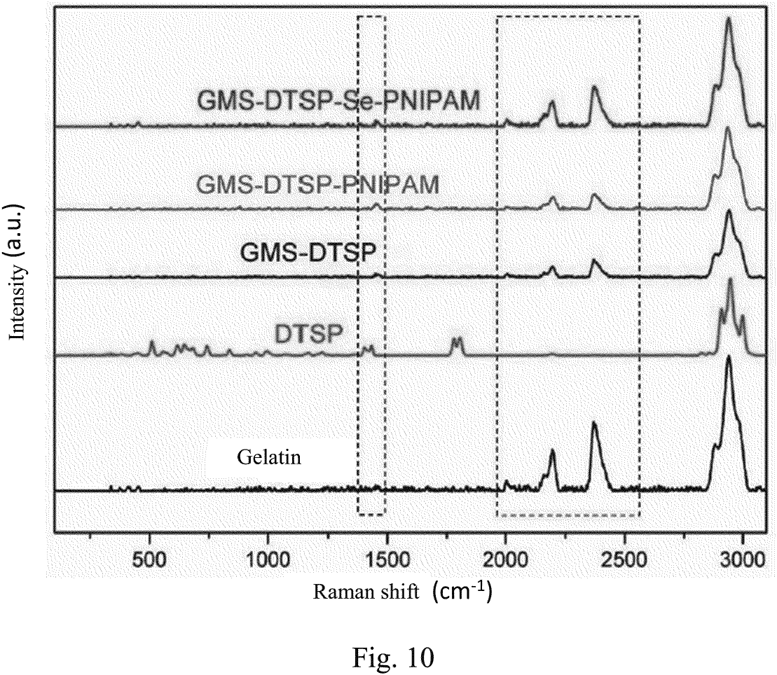

Since gelatin does not have a fixed structural formula, but there are a large number of hydroxyl groups and amine groups in the structure. The Raman peak cannot indicate the actual modified structure, but the difference between the microspheres can still be observed through the peak shift. As shown in FIG. 9, there are two signals in the range of 2000 to 2500 cm⁻¹ in the first group. After gelatin was crosslinked by glutaraldehyde (Gms), it can be confirmed that the two peaks in the three reaction stages are shifted to the left and right. FIG. 10 shows the second group of microspheres crosslinked with DTSP. It is observed that near 1400 cm⁻¹, the gelatin is crosslinked by DTSP, and each group of microspheres have this signal. It is inferred that DTSP indeed stabilized the gelatin crosslinking into microspheres. In addition, the two signals in the range of 2000 to 2500 cm⁻¹ are slightly changed.

### 3.2 FT-IR Analysis

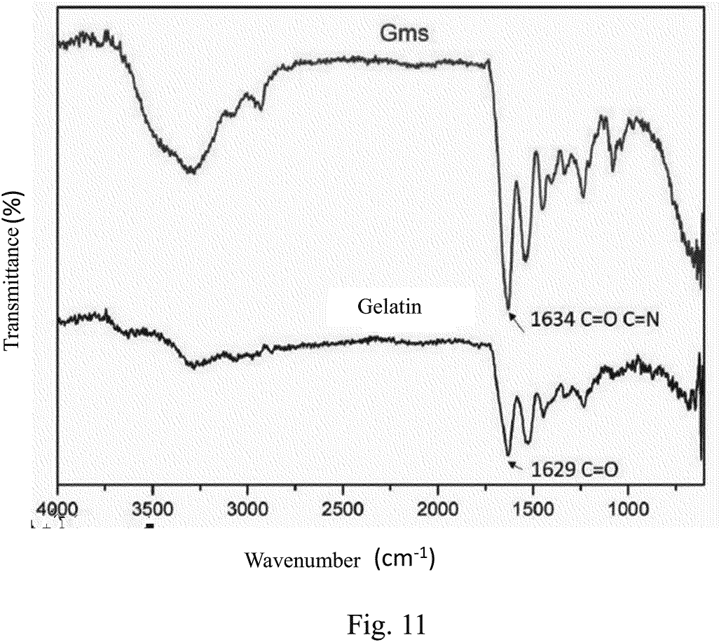

Raman spectroscopy is complementary to FT-IR, and is more suitable for detecting symmetrical bonds. The microspheres belong to an asymmetric structure, two groups of microspheres are analyzed by FT-IR, and there are relatively strong infrared absorption peaks. FIG. 11 shows that before gelatin was crosslinked, amide I was affected by C═O stretching vibration at 1629 cm⁻¹, and C═N stretching vibration of Schiff base reaction occurred at 1634 cm⁻¹. After crosslinking, gelatin was caused by C═O stretching of amide I and unreacted aldehyde group mixture. It is confirmed that the Schiff base reaction was formed between the amine group of gelatin and the carbonyl group (C═O) of glutaraldehyde, and the crosslinking of gelatin and glutaraldehyde was successful.

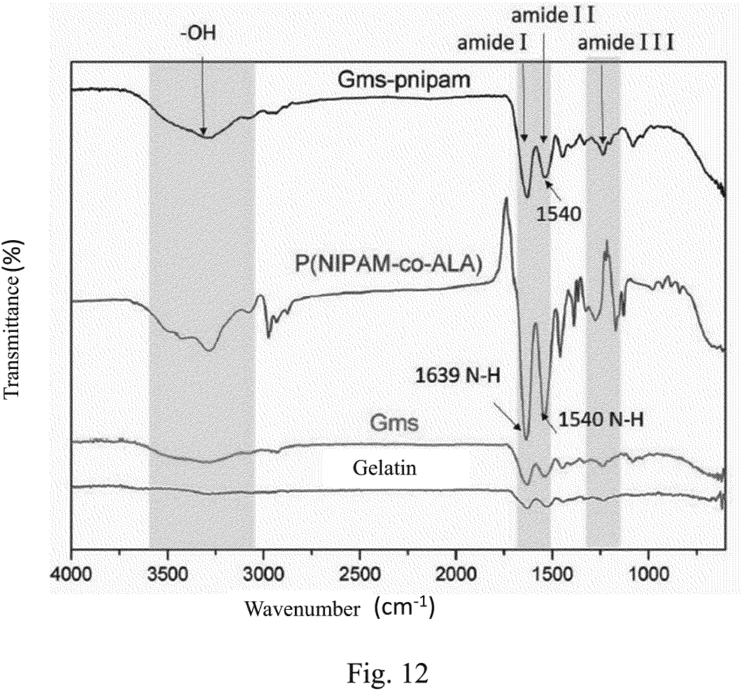

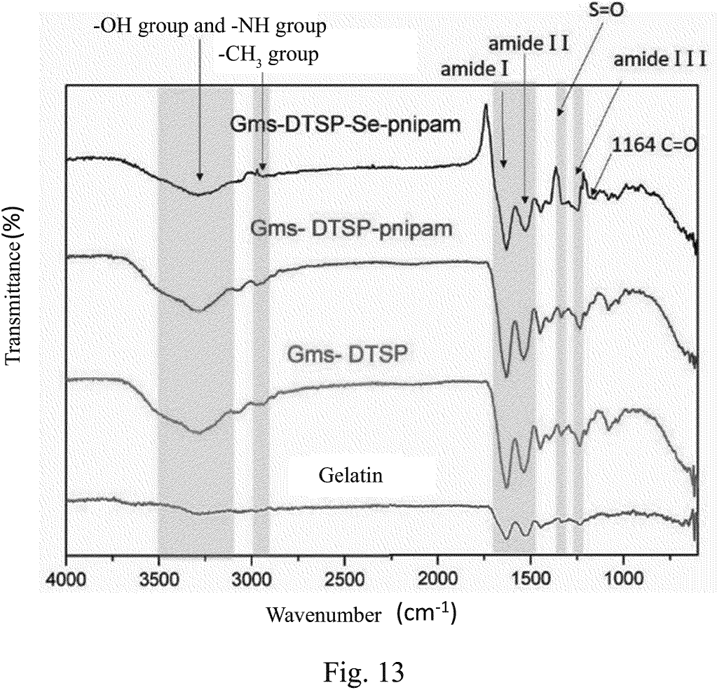

The main peaks in gelatin are amide I (stretching of C═O bond) at 1600 to 1700 cm⁻1, amide II (NH bending vibration and CN stretching vibration) at 1500 to 1590 cm⁻¹ and amide III around 1200 cm⁻¹ (NH bending vibration and CN stretching vibration), characteristic peaks are observed in both FIG. 12 and FIG. 13. In particular, peaks of O—H and N—H vibrations are observed at 3500 to 3000 cm⁻¹ using DTSP crosslinking agent, and symmetric stretching vibration peaks of —CH₃ group are observed at 2940 cm⁻¹. DTSP has an NHS active group, which reacts with the amine group (—NH₂) in the gelatin network to form a stable amide bond. At the same time, the NHS structure is released, and a characteristic peak of S═O is observed at 1390 cm⁻¹.

### 3.3 Scanning Electron Microscope (SEM) Analysis

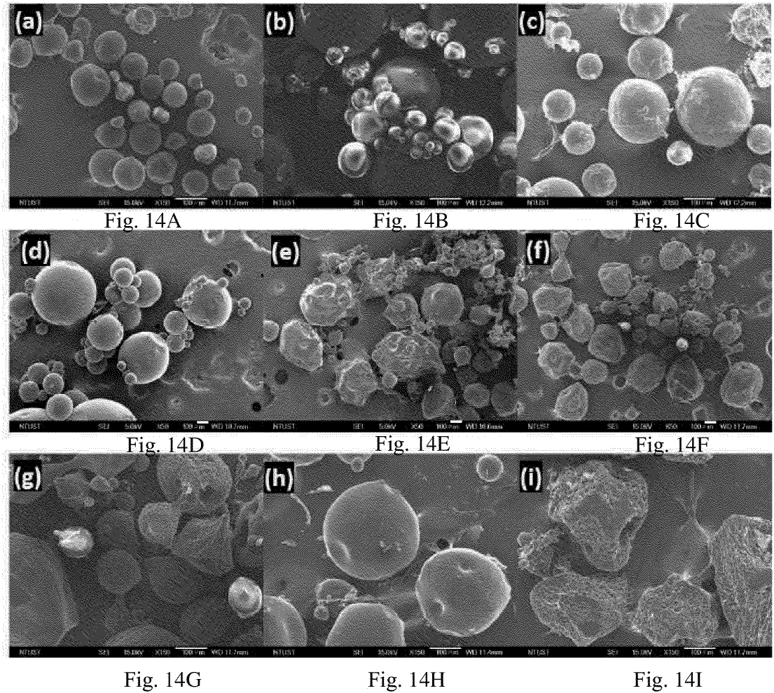

The size of the dried, cross-linked and solidified gelatin microspheres was analyzed by SEM and the surface morphology was observed. FIG. 14A shows microspheres obtained without crosslinking agent; FIG. 14B shows that the soluble microsphere (Gms) was crosslinked by glutaraldehyde to form agglomerates that cannot be sieved and have a non-uniform particle size range; FIG. 14C shows that after physically coated on the surface of the soluble-thermosensitive microsphere (Gms-pnipam), the microspheres have dispersibility, and the particles are obviously dispersed to improve the agglomeration phenomenon, and it is observed that the surface covered by thermosensitive polymer is formed in a powder state; FIG. 14D shows the use of DTPS crosslinking agent at 0.25 mM (Gms-0.25 mM DTSP); FIG. 14E shows the use of DTPS crosslinking agent at 0.6 mM (Gms-0.6 mM DTSP); FIG. 14F (50x magnification); FIG. 14G (150x magnification) shows DTPS crosslinking agent at 1.2 mM (Gms-1.2 mM DTSP), As the concentration of crosslinking agent increases, the surface of the microspheres changes from smooth to rough. Then the microspheres with thermosensitive polymer is observed: FIG. 14H shows the use of a physical coating (Gms-DTSP-pnipam), and it is found that the surface of the microspheres from the original roughness to a smooth state after being coated with thermosensitive polymer; another group of FIG. 14I shows chemical bonding (Gms-DTSP-Se-pnipam) using selenium crosslinking agent to chemically bond P(NIPAM-co-Allylamine) to the surface of gelatin, and there are obvious shrinkage holes.

In addition to SEM, energy-dispersive X-ray spectroscopy (EDS) was used at the same time to obtain the elemental analysis composition. The components of the microspheres crosslinked with glutaraldehyde are C, N, O elements and with the disulfide bond DTPS are C, N, O, S elements, and P (NIPAM-co-Allylamine) is chemically bonded to gelatin by the diselenide bond crosslinking agent to obtain C, N, O, S, Se elements. All of the above have crosslinking agent elements to confirm the successful crosslinking again. In order to understand the size of the microspheres in the dry state, Image J was used to analyze the particle size. Table 4 shows the average particle size of each group of gelatin microspheres.

TABLE 4

the average particle size of each group of gelatin microspheres

| Sample | Average diameter (µm) |
|---|---|
| (a) Gms | 142.19 ± 107.68 |
| (b) Gms-pnipam | 123.08 ± 51.75 |
| (c) Gms- 0.25 mM DTSP | 222.31 ± 169.13 |
| (d) Gms- 0.6 mM DTSP | 226.37 ± 69.25 |
| (e) Gms- 1.2 mM DTSP | 251.13 ± 81.14 |
| (f) Gms- DTSP-pnipam | 208.56 ± 85.96 |
| (g) Gms-DTSP-Se-pnipam | 230.17 ± 71.36 |

### 3.4 Particle Size Analysis in Swollen State

The microsphere particle size is defined in the range of 100 to 300 µm. First, gelatin microspheres smaller than 100 microns were separated using a mesh screen, and then immersed in warm medium for 5 days at 37° C. for simulating a real cell culture environment. Then an optical microscope was used to observe the swollen size and stability of the microspheres. Table 5 shows the particle size distribution of Gms and Gms-pnipam. When Gms is dried, the particle size pattern is agglomeration, which cannot be sieved for the first time, and the microspheres with small particles cannot be separated, and the size dispersion is large. After the surface coating of Gms-pnipam with P (NIPAM-co-Allylamine), the agglomeration phenomenon can be improved and it can pass through the sieve smoothly, and the size distribution of the microspheres is smaller.

TABLE 5

particle size of gelatin microspheres (Gms), (Gms-pnipam) after swelling

| Sample | Time (hrs.) | Average diameter (µm) |
|---|---|---|
| Gms | 0.5 | 160.6 50.5 |
| | 24 | 172.8 ± 57.5 |
| | 48 | 146.1 ± 78.3 |
| | 72 | 188.8 ± 62.3 |
| | 96 | 167.4 ± 82.4 |
| | 120 | 121.7 ± 52.3 |
| Gms-pnipam | 0.5 | 305.4 ± 20.3 |
| | 24 | 307.4 ± 22.5 |
| | 48 | 306.9 ± 26.2 |
| | 72 | 307.4 ± 26.8 |
| | 96 | 305.6 ± 28.4 |
| | 120 | 308.2 ± 25.3 |

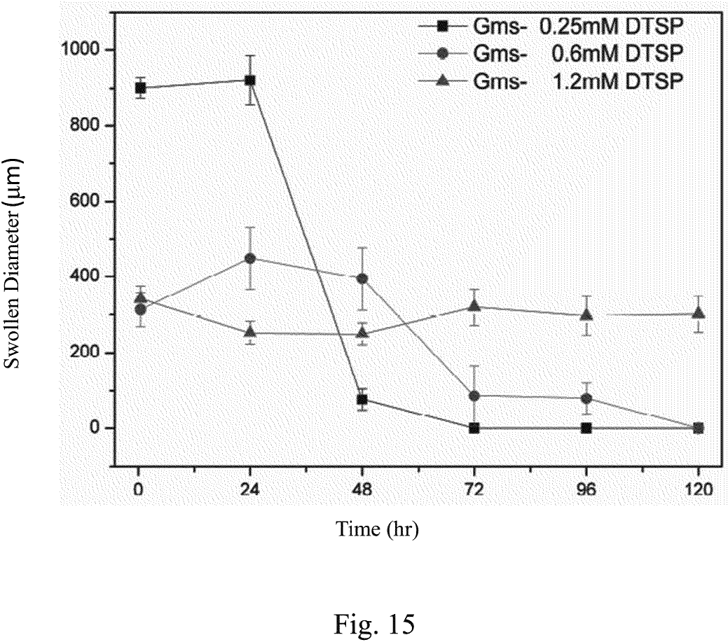

Another group was microspheres cross-linked with 0.25 mM, 0.6 mM, 1.2 mM DTSP. FIG. 15 and Table 6 show that the size of 0.25 mM DTSP microspheres swelled to 900 µm at 0.5 hrs. and gradually increased, and then the microspheres swelled to ruptured at 48 hrs. This group of crosslinking agents cannot stabilize the gelatin network, resulting in poor stability of the microspheres. 0.6 mM DTSP observed that the size was in the stable range within 48 hrs., the size of the microspheres was observed to decrease at 72 hrs., and slowly melted and disappeared with time. In contrast, the 1.2 mM DTSP crosslinked microspheres exhibited a smooth scaling curve within 120 hrs. with a particle size range of about 300 µm. It is confirmed that the concentration of the crosslinking agent is increased, and the microspheres with stable network size are obtained. The weight ratio of gelatin to 0.6-1.2 mM DTSP is 1.25:0.4 to 1.25:1 (1:0.32 to 1:0.8).

TABLE 6

Particle sizes of soluble-reduceable microspheres after swelling with different concentrations of crosslinking agent

| Sample | Time (hr.) | Average diameter (µm) |
|---|---|---|
| Gms 0.25 mM DTSP | 0.5 | 901.04 ± 29.3 |
| | 24 | 921.28 ± 65.4 |
| | 48 | 75.92 ± 29.2 |
| | 72 | - |
| | 96 | - |
| | 120 | - |
| Gms 0.6 mM DTSP | 0.5 | 313.26 ± 44.7 |
| | 24 | 448.67 ± 83.4 |
| | 48 | 394.83 ± 83.7 |
| | 72 | 85.29 ± 80.2 |
| | 96 | 78.36 ± 41.4 |
| | 120 | - |
| Gms 1.2 mM DTSP | 0.5 | 341.30 ± 33.3 |
| | 24 | 252.76 ± 30.5 |
| | 48 | 250.02 ± 29.3 |
| | 72 | 319.82 ± 47.3 |
| | 96 | 297.14 ± 51.1 |
| | 120 | 301.75 ± 47.7 |

Table 7 is the Gms-DTSP-pnipam and Gms-DTSP-Se-pnipam microspheres formed after using Gms-1.2 mM DTSP combined with thermosensitive polymer P (NIPAM-co-Allylamine). After 120 hrs. observation, dimension of the microspheres stability changes without cracking and swelling phenomenon. Therefore, these three groups are suitable for cell culture experiments, which confirmed the successful synthesis of soluble-reduceable-thermosensitive gelatin microspheres.

TABLE 7

Particle size of soluble-reduceable-thermosensitive microspheres after swelling

| Sample | Time (hr.) | Average diameter (µm) |
|---|---|---|
| Gms-DTSP | 0.5 | 341.30 ± 33.3 |
| | 24 | 252.76 ± 30.5 |
| | 48 | 250.02 ± 29.3 |
| | 72 | 319.82 ± 47.3 |
| | 96 | 297.14 ± 51.1 |
| | 120 | 301.75 ± 47.7 |
| Gms-DTSP -pnipam | 0.5 | 247.28 ±34.9 |
| | 24 | 279.25 ± 37.9 |
| | 48 | 306.70 ± 37.3 |
| | 72 | 288.74 ±37.1 |
| | 96 | 333.61 ± 41.4 |
| | 120 | 367.94 ± 45.1 |
| Gms-DTSP -Se-pnipam | 0.5 | 202.76 ± 32.3 |
| | 24 | 244.49 ± 32.5 |
| | 48 | 254.65 ± 34.3 |
| | 72 | 271.31 ± 30.2 |
| | 96 | 291.28 ± 30.1 |
| | 120 | 239.60 ± 47.7 |

### 3.5 Microsphere Swelling Test

10 mg of dry weight microspheres (W1) were placed in a 15 mL centrifuge tube and immersed in a 37° C. medium, and the medium was removed at various time points. Lens tissue was used to remove excess liquid, wet sample was left for weighing (W3) and repeated three times per group. Microsphere swelling ratio calculation formula is:

$$\text{Swelling} \text{ratio =} \left(\text{W3-W2}\right) / \text{W1}$$

The formula for calculating water content is:

$$\text{Water content} \left(\%\right) = \left[1 \text{-W1} / \left(\text{W3-W2}\right)\right] \times 100$$

W1: dry sample weight ; W2: dry sample and centrifuge tube weight ; W3: wet sample and centrifuge tube weight.

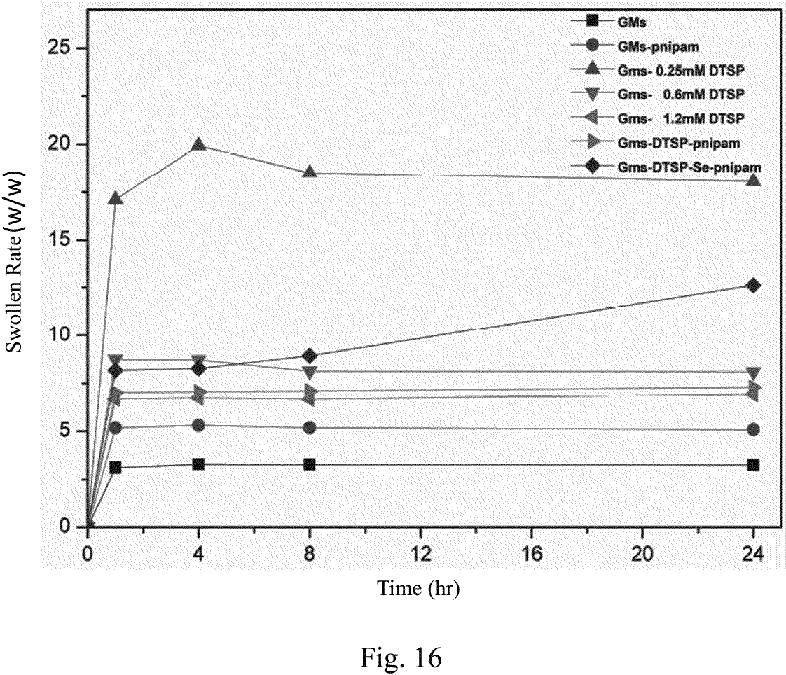

The swelling degrees of microspheres were compared and used to evaluate the water absorption properties of polymer microspheres at 37° C. In the experiment, the dried microspheres were immersed in the medium for 24 hrs., and the weight after swelling was measured. FIG. 16 shows the swelling properties of different gelatin microspheres. It is observed that the swelling degree decreased with the increase of 0.6 mM and 1.2 mM DTSP concentrations, and the swelling ratio of 0.25 mM DTSP reached 18.05 within 24 hrs. However, as shown in the aforementioned Table 6, the microspheres swelled and ruptured at 48 hrs., and this group of crosslinking agents could not stabilize the gelatin network. The physical coating of the surface thermosensitive polymer increases the degree of swelling and increases the water content at the same time. Adding P(NIPAM-co-Allylamine) into gelatin microspheres can significantly improve the water absorption capacity, and the most obvious is GMS-DTSP-Se-pnipam microspheres with a swelling ratio of 12.62 ± 0.07 and a water content of 93.32 ± 0.97%.

TABLE 8

Swelling ratio and water content of different microspheres after swelling

| Sample | Swelling ratio(w/w) | Water content (%) |
|---|---|---|
| Gms | 3.24±0.05 | 67.14±0.26 |
| Gms-pnipam | 4.59±0.43 | 80.82±1.27 |
| Gms-0.25 mM DTSP | 18.05±1.04 | 93.57±1.88 |
| Gms-0.60 mM DTSP | 8.08±0.61 | 88.95±0.76 |
| Gms-1.20 mM DTSP | 6.94±0.17 | 89.13±2.71 |
| Gms-DTSP-pnipam | 7.29±0.31 | 90.02±0.57 |
| Gms-DTSP-Se-pnipam | 12.62±0.07 | 93.32±0.97 |

### 3.6 Degradation Behavior of Dissolved Microspheres

Disulfide bond (—S—S—) is usually formed by the natural crosslinking of two sulfur atoms between the side groups of cysteine. Alkylation of free cysteine with the chemical reducing agent DTT is used to reduce disulfide bonds, resulting in cleavage of di-cysteine into cysteine, and diselenide bonds have a similar redox mechanism to disulfide bonds. The oxidation responsiveness of diselenide bonds is greater than that of disulfide bonds. Selenium has a large atomic radius and weak electronegativity, and its lower bond energy can be easily reduced to break the diselenide bond to generate a selenate intermediate (RSe⁻).

The present disclosure provides a degradation experiment for the oxidative dissolving microspheres. In order to simulate the cell culture environment, Gms-DTSP, Gms-DTSP-pnipam, Gms-DTSP-Se-pnipam were immersed in the medium and maintained at 37° C. Subsequently, 25 mM reducing agent DTT was added to observe the changes in the degradation of the gelatin polymer network. Table 9 shows that Gms-DTSP-Se-pnipam disappeared within 15 minutes, leaving only a clear solution, and the remaining two groups completely degraded within 30 minutes.

TABLE 9

Degradation behavior of reduced microspheres in 25 mM DTT

| Sample | Degradation time (min) |
|---|---|
| Gms-DTSP | 30 |
| Gms-DTSP-pnipam | 30 |
| Gms-DTSP-Se-pnipam | 15 |

### Example 4 Cell Viability

### 4.1 Sensitive Block Copolymer

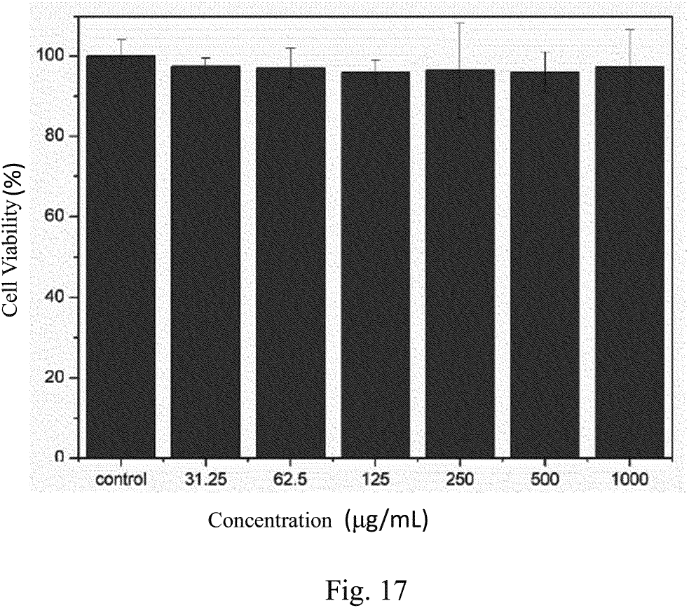

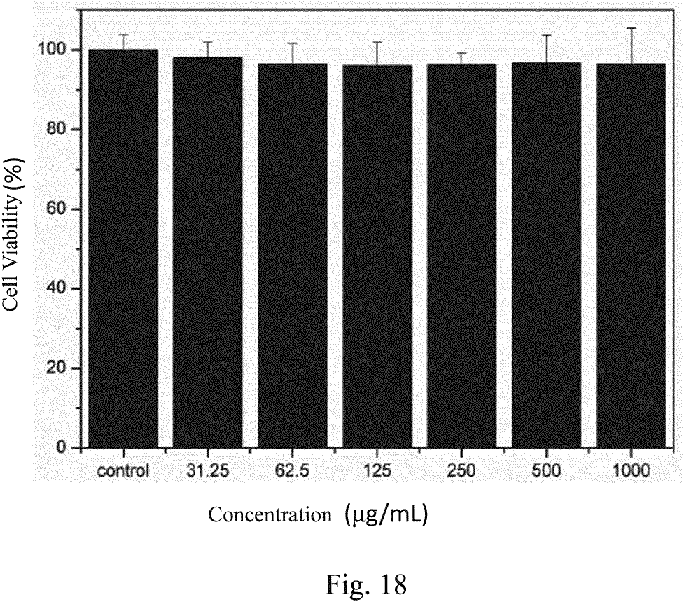

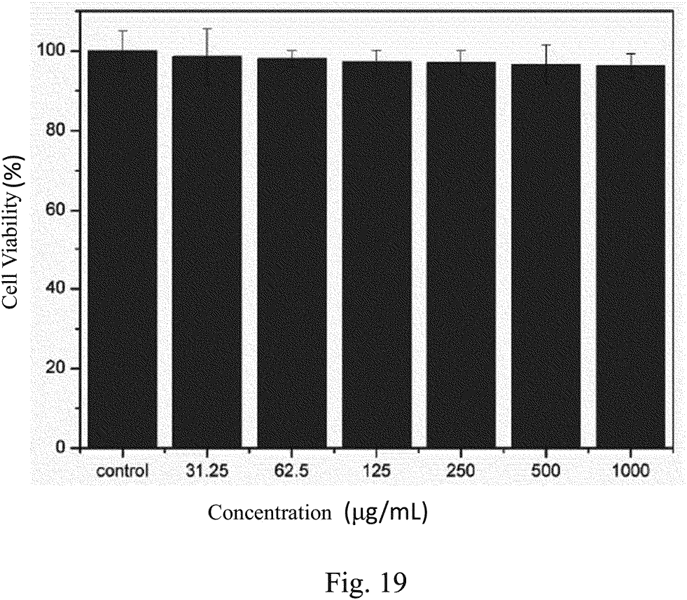

FIG. 17, FIG. 18, FIG. 19 show that 1% ALA, 3% ALA, 5% ALA thermosensitive polymer were co-cultured with MDCK cells for 24 hrs. The cell viability of the three copolymers were 97.4%, 96.5%, and 96.3% at the highest concentration of 1000 ug/mL, respectively. This confirms that the thermosensitive polymer has good biocompatibility with MDCK cells (a standard mammalian cell line in biomedical research). When cells were stimulated by toxic substances, it caused cell morphological changes and led to apoptosis. MTT (3-[4,5-dimethylthiazol-2-yl]-2,5 diphenyl tetrazolium bromide) interacted with the mitochondrial succinate dehydrogenase of living cells through the MTT assay, and purple crystals were produced under the reduction reaction. The number of viable cells was qualitatively obtained by absorbance detection using a disc spectrometer at 570 nm.

### 4.2 Gelatin Microsphere

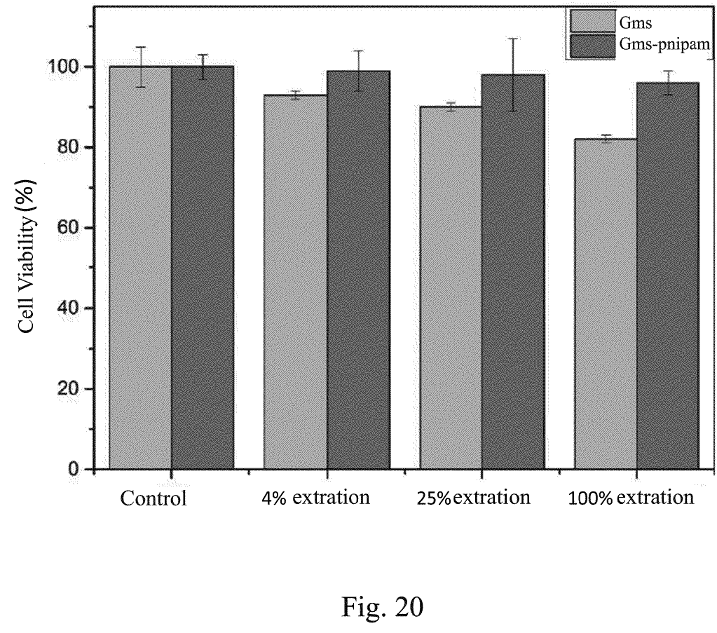

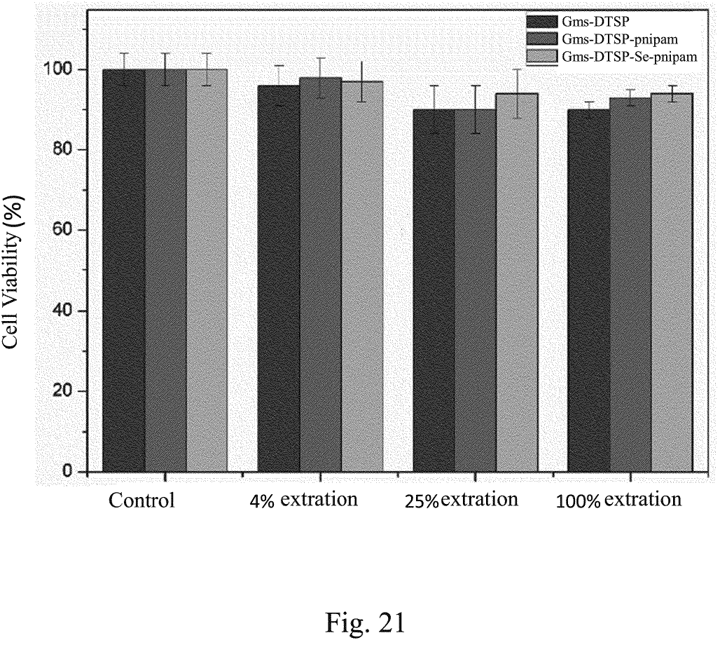

Microspheres are carriers and cannot be dissolved in the medium, and the shapes of microspheres are not uniform. According to ISO-10993, the microspheres were immersed in 37° C. medium at 0.1 g/mL for 24 hrs., and the medium after different percentages of immersed microspheres were extracted and co-cultured with MDCK for 24 hrs. FIG. 20 shows that the dissolvable microspheres cross-linked with glutaraldehyde have a cell viability of 82% at the highest concentration of 0.1 g/mL. The cell viability is improved by 96.3% at the highest concentration of Gms-pnipam. The thermosensitive polymer has a great biocompatibility because of the physical coating of thermosensitive polymer on the surface. FIG. 21 shows another group of soluble-reduceable microspheres crosslinked with DTSP. The three types of microspheres also have good biocompatibility, all of which can be greater than 90%. At the highest concentration, the cell viability also reaches 94%, which confirms that each group of microspheres does not have the ability to kill cells, which is beneficial to the subsequent adherence culture experiments.

### Example 5 Cell Attachment and Detachment Test

### 5.1 Thermosensitive Polymer Cell Attachment and Detachment Test

Cells was grouped according to 1% ALA, 3% ALA, 5% ALA in the experiment of attachment and detachment of P(NIPAM-co-Allylamine). After spin coating on plastic coverslips, each group was co-cultured with 0.5x10⁶ cells/mL MDCK. Observation under light microscope at 37° C., cells adhered to P(NIPAM-co-Allylamine) membrane and grew in a tightly flattened cluster shape, fully exhibiting good adhesion characteristics (not shown). When the temperature dropped to 4° C., the hydrophilic groups on the surface of the copolymer membrane interacted with the charged groups of the cells, resulting the cells to float in the form of lumps and suspended in the PBS solution. In addition, a blank plastic cover glass (without temperature-sensitive block copolymer coating) was used as a control group. When the temperature was adjusted to a low temperature, the morphology of the cells did not change and did not detach, and the cells still adhered to the cover slip (not shown).

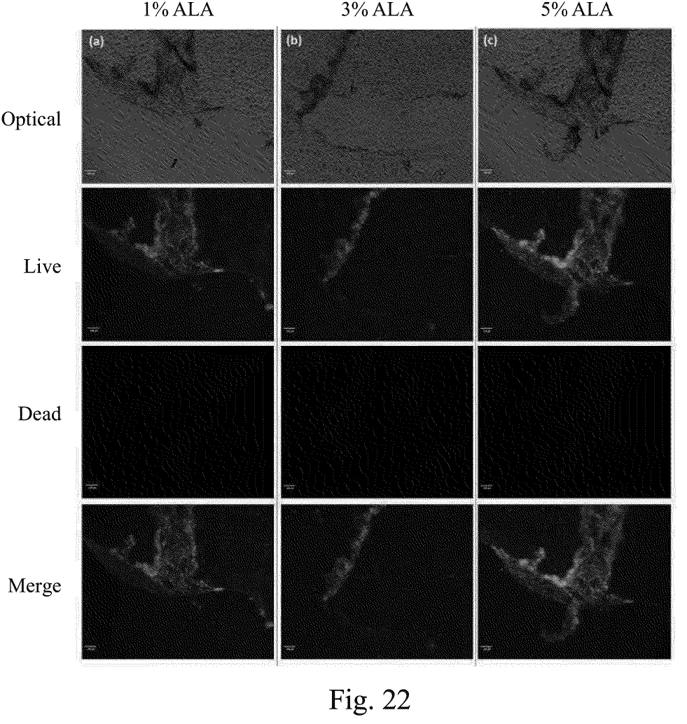

Cells detached via temperature were stained for viability using live/dead cell imaging reagents. Live cells can easily penetrate into live cell membranes through Calcein-AM calcein. After hydrolysis, calcein remained in the cells and emitted strong green fluorescence, while dead cells were subjected to BOBO™-3 Iodide to penetrate the damaged cell membrane and stain the nucleus to release red fluorescent. FIG. 22 shows that the cells detached from the three groups of P(NIPAM-co-Allylamine) membranes are observed under a fluorescence microscope. The proportion of green fluorescence in the overlapped images is mostly green, which proves that the temperature-induced detached cells still maintain good viability.

The cells are detached from the thermosensitive surface P(NIPAM-co-Allylamine) by using the simple method of temperature regulation, and the cells are not harmed and the doubts of damage caused by the traditional enzymatic desorption method are avoided at the same time. From the perspective of engineering medicine, cells are cut off the connection between cells by traditional enzymatic hydrolysis, and the harvested cells are independent single cells, which cannot regenerate continuous cell sheets. The thermosensitive gel layer can obtain cell sheets with complete intercellular connections, which belongs to non-invasive desorption. The cells after confluent culture need to lower the temperature to successfully harvest cell sheets with tissue structures, thereby achieving the purpose of biomedical tissue regeneration and repair.

### 5.2 Temperature-induced Detachment Behavior of Thermosensitive Microspheres

Regarding thermosensitive polymer P(NIPAM-co-Allylamine), the concentration of ALA will increase the amine group (—NH₂) in the structure, which will increase the positive charge and hydrophilicity. The cell culture method is the same as described in 5.1 above. For the copolymer of 5% ALA, the cells had a stickier culture surface upon attachment. In the case of cell detachment at low temperature, the desorption time of 5% ALA was longer than that of the other two groups, indicating that more ALA content is helpful for cell adhesion, but too much ALA monomer embedded in the copolymer will make the copolymer too hydrophilic and lose temperature sensing characteristic. Therefore, in the preparation of thermosensitive gelatin microspheres, 5% ALA copolymer was finally selected for thermosensitive modification on the surface of gelatin microspheres through the above evaluation of cell adsorption and desorption.

In order to detect the temperature-induced cell detachment ability of P(NIPAM-co-Allylamine) on the microspheres, the detachment behaviors of Gms-pnipam and Gms-DTSP-pnipam were first tested. The gelatin microspheres induced cell detachment by temperature. After 30 and 60 minutes of desorption at a low temperature of 4° C., it can be seen that the cells only dropped slightly (not shown), which confirms that the gelatin microspheres use temperature-induced cell detachment is less effective and takes longer. Therefore, it is necessary to use traditional enzymatic hydrolysis and degradation methods to improve the efficiency of cell desorption from the carrier.

### 5.3 Attachment Detachment Test of Soluble Microsphere Cells

Observing with a fluorescence microscope, Hoechst 33342 was used to observe whether the cells adhered to the surface of the microspheres for growth. Hoechst 33342 penetrated the cell membrane and emitted blue fluorescence. It was confirmed by the fluorescence microscopy that the blue cells were attached to five types of microspheres (including Gms, Gms-pnipam, Gms-DTSP, Gms-DTSP-pnipam, Gms-DTSP-Se-pnipam, not shown) for the subsequent evaluation of the ability of the five microspheres to attach cells.

In order to evaluate the microsphere cell attachment and detachment test, after the Gms, Gms-pnipam was swollen, each group was cultured with 0.5 × 10 cells/mL MDCK cells, and the attachment of MDCK to the microspheres was observed at 1, 3, 5, 7, 24 hrs. with an optical microscope, and the cell attachment rate was calculated. The results showed that cells began to adhere to the edge of the microspheres within 1 hrs., but the size of Gms was not uniform because the agglomeration of the microspheres. The microspheres with small size were difficult to observe whether there were adherent cells or not. The size of Gms-pnipam was relatively uniform, and it can be seen that the cells were obviously attached. Therefore, the cell adhesion effect of Gms-pnipam is better than that before modification, so pnipam is benefit to cell adhesion (not shown). In addition, the results also shows that the trypsinization method completely performed the cell detachment experiment. The microspheres were immersed in trypsin and observed after 5 minutes at 37° C., and cells were dropped from the surface of Gms. With the increase of time, trypsin can break the peptide bond formed by lysine or arginine, and the cell detachment effect increases with trypsin digestion. After 10 minutes, the microspheres were washed with PBS to retrieve the cells. After washing, there were still many cells remaining on the surface of the microspheres, and the detachment effect was not good. Another group of Gms-pnipam was treated with trypsin for 5 minutes, and it could be seen that the cells and thermosensitive polymer were cut off at the same time. After 10 minutes, it could be seen that the cells had almost completely fallen into suspension. After washing the microspheres with PBS, the cells were retrieved and the surface of the microspheres tended to be smooth and flat. It is confirmed that the thermosensitive polymer contributes to the cell detachment effect (not shown).

In addition, the Gms, Gms-pnipam attached with the cells were added with live/dead stains to identify the degree of cell death on the surface of gelatin microspheres. Calcein-AM staining live cells elicited green fluorescence, while BOBO-3 Iodide staining dead cells elicited red fluorescence. The results shows that a large number of green fluorescent cells and a few dead cells attached to the microspheres, and the cells detached with trypsin for too long damaged membrane proteins. In addition, the cells detached from Gms-pnipam showed green fluorescence, and red cells accounted for the minority. Cells were not completely detached from Gms after 10 minutes, and many healthy cells were still attached to the microspheres. Therefore, it is proved that Gms has the ability to attach cells, but the physical coating of thermosensitive polymer on the surface of the microspheres can enhance the detachment ability of the carrier to release cells.

### 5.4 Cell Attachment Rate and Detachment of Soluble Microsphere

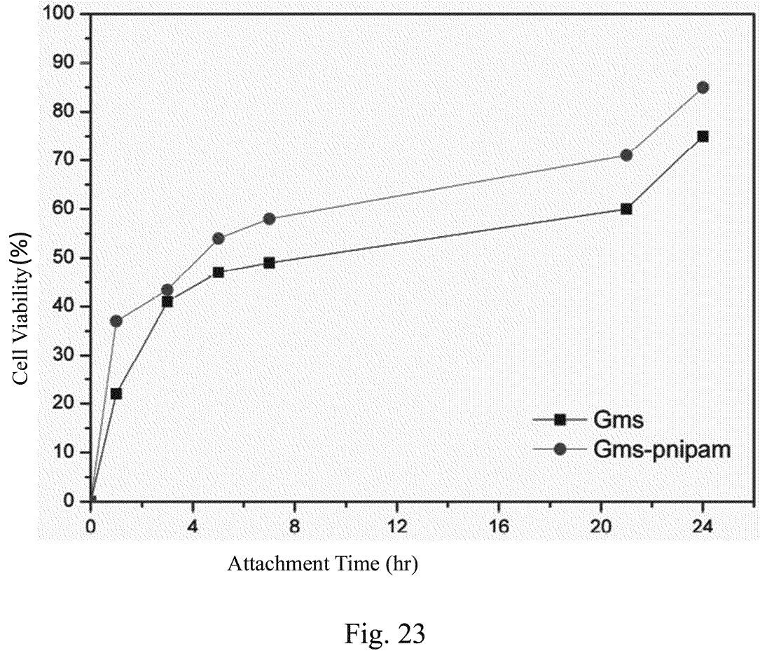

FIG. 23 shows the ability of microspheres Gms, Gms-pnipam to attach cells. After 1 hr. incubation, Gms-pnipam reached about 40% cell attachment rate, Gms was 23% cell attachment rate, and Gms and Gms-pnipam respectively were 74% and 85% after 24 hrs. incubation. After the surface was coated with thermosensitive polymer, the attachment rate was increased by 11%. The gelatin microspheres Gms, Gms-pnipam detachment properties were then evaluated. Gms and Gms-pnipam, as mentioned above in Section 5.3, were digested with trypsin for the same time, and the detachment efficiency of Gms was lower than that of Gms-pnipam. After the detached cells being washed, centrifugated, and calculated, the number of cells harvested from Gms and Gms-pnipam were 192,500 cells/mL and 355,750 cells/mL, respectively. After the surface was coated with thermosensitive polymer, the detachment ratio increased by 45.8%.

### 5.5 Cell Attachment and Detachment of Soluble-reduceable Microsphere

In the present disclosure, a crosslinking agent DTSP with redox properties of disulfide bond structure was used in the manufacture of microspheres. Gms-DTSP, Gms-DTSP-pnipam, Gms-DTSP-Se-pnipam were synthesized and cultured for two days, and the attachment of MDCK cells to the microspheres was observed by light microscope.

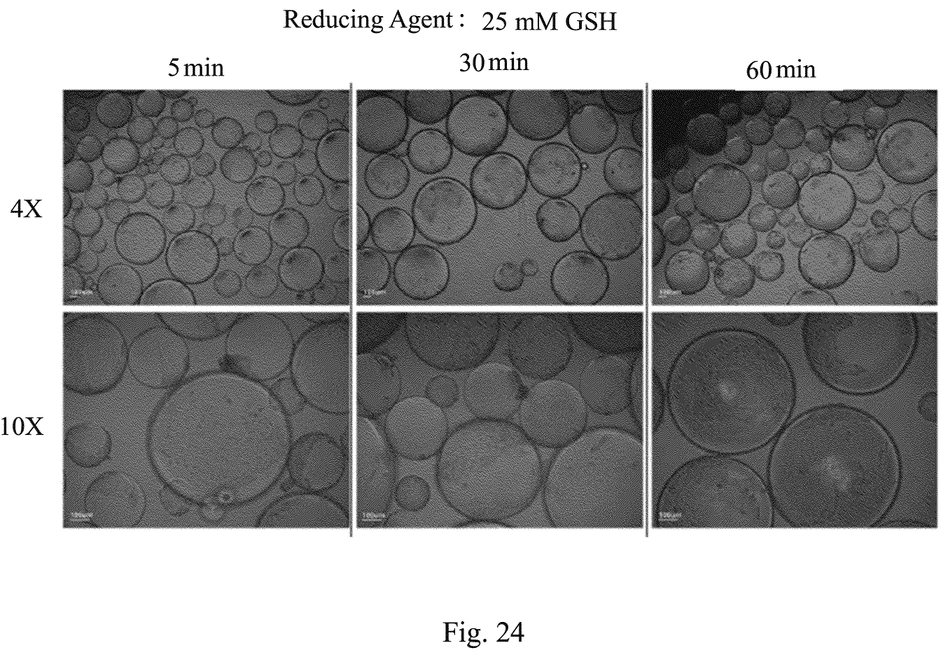

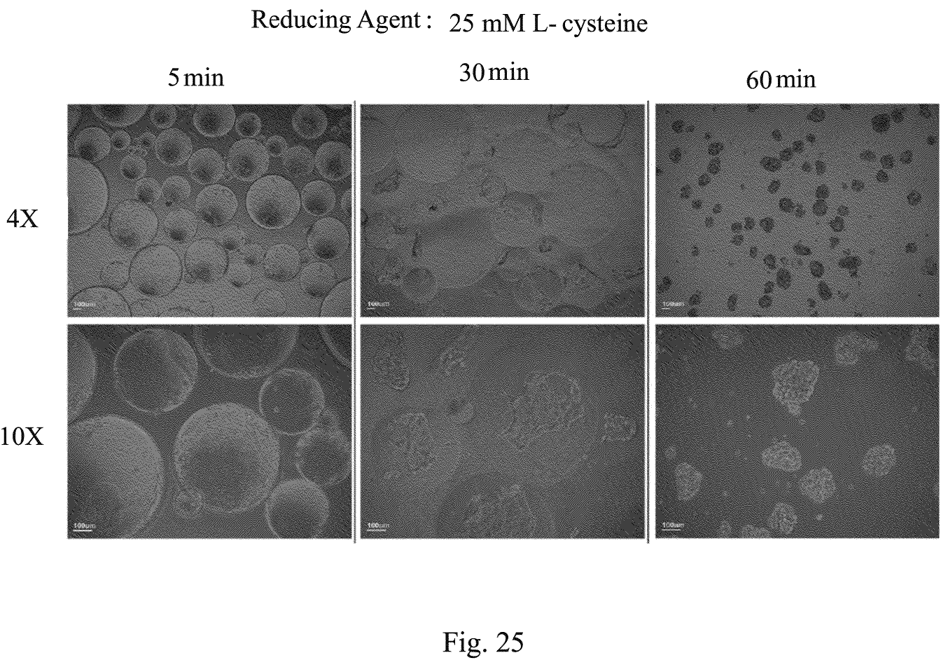

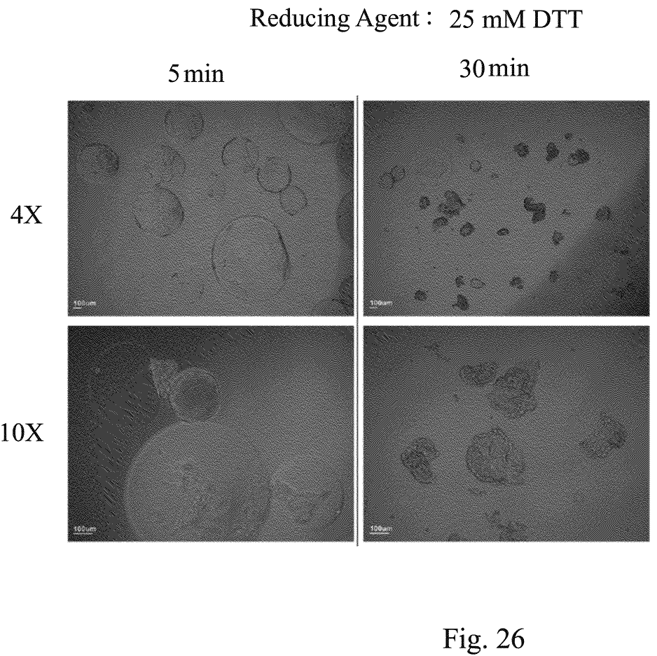

The degradation behavior of reduced gelatin microspheres, Gms-DTSP-pnipam, was evaluated using three reducing agents (GSH, L-cysteine (L-cysteine), and DTT) all at a concentration of 25 mM, and the condition of cell detachment was observed. FIG. 24 shows that the microspheres did not burst, dissolve and other changes after using the GSH reducing agent for 60 minutes. FIG. 25 shows that the microspheres swelled and the cells fell off in clumps after 30 minutes of using L-cysteine reducing agent, and the microspheres completely dissolved and disappeared after 60 minutes. FIG. 26 shows that the swollen and deformed microspheres were rapidly degraded after 5 minutes using the DTT reducing agent, and only clumps of cells remained at 30 minutes. Among the three reducing agents tested, DTT containing dithiols showed the best effect in cutting disulfide bonds and had a higher redox potential than GSH and L-cysteine. GSH and L-cysteine are monothiols that require other thiol-containing molecular catalysis to accelerate the reduction of disulfide bond cleavage.

Gms-DTSP and Gms-DTSP-pnipam needed 30 minutes to degrade the microspheres with DTT reducing agent. In particular, the soluble type of Gms-DTSP belonged to DTT bursting of microspheres, and the cross-linked network was cut off by the reducing agent, so that the spheres ruptured and the cells finally became single particles. The degradation pattern of surface-coated Gms-DTSP-pnipam microspheres is that cells were detached by DTT in clump. It is inferred that the protection of the outer thermosensitive polymer coating caused the coating and cells to peel off from the outside, so that the spheroid itself could slowly degrade. The final cells were in the form of clumps, and the agglomerated cells were peeled off together with the thermosensitive polymer coating (not shown).

In addition, thermosensitive polymer coating was hydrophilic at low temperature. Gms-DTSP-pnipam gradually disintegrated after 5 minutes at low temperature (such as 4° C.), and cells became dispersed in 10 minutes. Gms-DTSP-Se-pnipam had the shortest disintegration time (not shown).

Gms-DTSP, Gms-DTSP-pnipam, Gms-DTSP-Se-pnipam with attached cells were added live/dead cell imaging reagents to identify the degree of cell death on the surface of gelatin microspheres. The results shows that the microspheres were encapsulated by a large number of green fluorescent cells, and the detached cells were excited to emit green fluorescence after detachment (not shown in the figure), which proved that the reduced gelatin microspheres have the ability to attach cells, and the cells still survive when detachment.

### 5.6 Cell Attachment Rate and Detachment of Soluble Microsphere

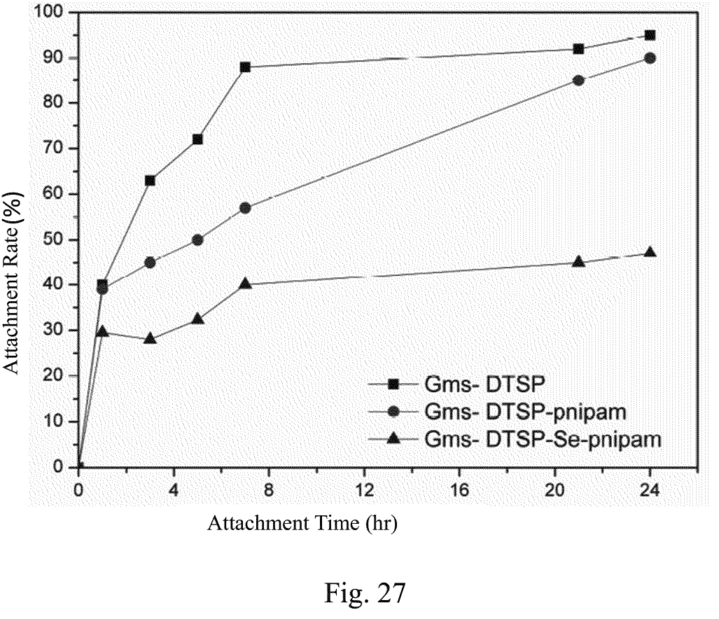

FIG. 27 shows the ability of soluble-reduceable and soluble-reduceable-thermosensitive microspheres Gms-DTSP, Gms-DTSP-pnipam, Gms-DTSP-Se-pnipam to adsorb cells. The cell attachment rates of Gms-DTSP, Gms-DTSP-pnipam, and Gms-DTSP-Se-pnipam were 40%, 39%, and 29.5% after 1 hrs. of culture; Gms-DTSP first reached 88% after 8 hrs.; Gms-DTSP, Gms-DTSP-pnipam, and Gms-DTSP-Se-pnipam cell attachment rates were 95%, 90%, and 47% after 24 hrs., respectively. It is observed from the SEM of Example 3.3 that the microspheres have tiny concave-convex holes on the surface to increase cell adhesion, and the ability of Gms-DTSP-pnipam to attach cells is better than that of Gms-DTSP-Se-pnipam.

### 5.7 Re-culture Test of Harvested Cells

To demonstrate the viability and subsequent medical applications of cells harvested using gelatin microspheres, cells detached from Gms, Gms-pnipam, Gms-DTSP, Gms-DTSP-pnipam, Gms-DTSP-Se-pnipam were washed and centrifuged, and then placed in a petri dish for recultivation. The results shows that the cells cultured with Gms, Gms-DTSP-Se-pnipam after one day grow slower because fewer cells are recollected from the original. The rest of Gms-pnipam, Gms-DTSP, Gms-DTSP-pnipam are all in a state of 80% full, and the cells grew in a densely flattened and clustered form, showing good cell viability (not shown). Therefore, the cells detached from the microspheres of the present disclosure have the ability of recultivation.

The soluble-reduceable microcarrier of the present disclosure facilitates cell attachment by reducing crosslinking agent, and facilitates cell detachment by using a reducing agent. In some embodiments, the cells were detached within 30 minutes of using the reducing agent, and the cells were viable cells after detachment, demonstrating that this method is not toxic.

The soluble-thermosensitive microcarrier of the present disclosure facilitates cell attachment when the temperature is higher than LCST through the coating of thermosensitive polymer, and the soluble-thermosensitive microcarrier facilitates cell detachment when the temperature is lower than LCST. In some embodiments, the spherical shape becomes complete after immersing in the medium, and the particle size is stably controlled between 280 µm to 350 µm. At the same time, the surface of the microspheres is protected by thermosensitive polymer, which helps the stability of the microspheres at 37° C. In some embodiments, cell attachment rate increases by 11% through the modification of thermosensitive polymer, and the cell detachment rate increases by 45.8% through the enzymatic hydrolysis method, thereby effectively improving the attachment and detachment capabilities of the microcarrier.

.While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A soluble microcarrier, comprising:
a soluble polymer comprising a plurality of soluble monomers binding to each other with a reducing crosslinking agent.

2. The soluble microcarrier of claim 1, wherein the reducing crosslinking agent comprises binding to a hydroxyl group, an amine group, a thiol group, or a carboxylic acid group of the soluble polymer.

3. The soluble microcarrier of claim 1, wherein the reducing crosslinking agent comprises disulfide bond crosslinking agent, or diselenide bond crosslinking agent.

4. The soluble microcarrier of claim 3, wherein the disulfide bond crosslinking agent comprises 3,3′-dithiodipropionic acid di (N-hydroxysuccinimide ester) (DTSP), 3,3′-dithiobis(sulfosuccinimidylpropionate (DTSSP)), cysteine, or dithiobis (succinimidyl propionate)(DSP).

5. The soluble microcarrier of claim 3, wherein the diselenide bond crosslinking agent comprises 3,3′-Dithiodipropionic acid di(N-hydroxysuccinimide ester), 3,3′-diselanediyldipropionic acid, 2,2′-diselanediylbis(ethan-1-amine), 2,2′-diselanediylbis(ethan-1-ol), or a combination thereof.

6. The soluble microcarrier of claim 1, wherein the soluble polymer comprises cellulose, collagen, gelatin, sodium alginate, chitosan, hyaluronic acid, fruit acid, or a combination thereof.

7. The soluble microcarrier of claim 1, wherein a weight ratio of the soluble polymer and the reducing crosslinking agent is from 1:0.08 to 1:0.8.

8. The soluble microcarrier of claim 1, further comprising a thermosensitive polymer encompassing the soluble polymer.

9. The soluble microcarrier of claim 8, wherein the thermosensitive polymer comprising poly(N-isopropylacrylamide)(PNIPAM), poly(N,N diethylacrylamide)(PDEAAM), poly(N-vinylcaprolactam)(PVCL), poly(2-isopropyl-2-oxazoline)(PIOZ), poloxamer, or a combination thereof.

10. The soluble microcarrier of claim 9, wherein the thermosensitive polymer further comprises acrylic acid (AAC), allylamine (ALA), acrylamide (AAm), [2-(Methacryloyloxy)ethyl]dimethyl-(3-sulfopropyl)ammonium hydroxide (DMAPS), 2-(Diethylamino)ethyl methacrylate (DEAEMA), 2-Hydroxyethyl methacrylate (HEMA), or a combination thereof.

11. The soluble microcarrier of claim 10, wherein the thermosensitive polymer is poly(N-isopropylacrylamide)-co allylamine (P(NIPAM-co-ALA)).

12. The soluble microcarrier of claim 11, wherein a weight percentage of the ALA to P(NIPAM-co-ALA) is from 1% to 15%.

13. The soluble microcarrier of claim 8, wherein the thermosensitive polymer is bound to an outer surface of the soluble polymer by the reducing crosslinking agent.

14. The soluble microcarrier of claim 8, wherein the thermosensitive polymer is physically bound to an outer surface of the soluble polymer.

15. A method for manufacturing a soluble microcarrier, comprising steps of:
providing a soluble polymer; and
processing the soluble polymer and a reducing crosslinking agent with a mixing process, wherein when the soluble polymer is in contact with the reducing crosslinking agent, crosslinking occurs to obtain the soluble microcarrier.

16. The method of claim 15, wherein the step of providing the soluble polymer comprises:
heating a plurality of soluble monomers to a liquid state;
mixing an oil and a surfactant to obtain a mixed solution;
mixing the mixed solution and the plurality of soluble monomers to obtain a water-in-oil emulsion; and
cooling the water-in-oil emulsion to shape to obtain the soluble polymer.

17. The method of claim 15, further comprising:
providing a thermosensitive polymer; and
mixing the soluble microcarrier and the thermosensitive polymer to obtain a soluble-thermosensitive microcarrier.

18. The method of claim 15, wherein the mixing process comprises microfluidics, titration, electrospinning, emulsion polymerization, film emulsification, or a combination thereof.

19. A method of using a soluble microcarrier as claimed in claim 1, wherein when the soluble microcarrier is in contact with a reducing agent, the soluble microcarrier degrades.

20. A method of using a soluble microcarrier as claimed in claim 8, wherein when the soluble microcarrier is in contact with a reducing agent, contacts a lower critical solution temperature, contacts the reducing agent and then contacts the lower critical solution temperature, or contacts the lower critical solution temperature and then contacts the reducing agent, the soluble microcarrier degrades.
